(19) United States
(12) Patent Application Publication
(10) Pub. No.: US 2025/0295289 A1
(43) Pub. Date: Sep. 25, 2025

(54) SYSTEMS AND METHODS FOR ROBOTIC ENDOSCOPE SYSTEM UTILIZING TOMOSYNTHESIS AND AUGMENTED FLUOROSCOPY

(71) Applicant: Noah Medical Corporation, San Carlos, CA (US)

(72) Inventors: Tao Zhao, Sunnyvale, CA (US); Zhongming Shen, Sunnyvale, CA (US); Changjiang Yang, Dayton, MD (US); Pei Han, Redwood City, CA (US); Michael Ian Larkin, Santa Barbara, CA (US)

(21) Appl. No.: 19/082,681

(22) Filed: Mar. 18, 2025

Related U.S. Application Data
(63) Continuation of application No. PCT/US2023/079481, filed on Nov. 13, 2023.
(60) Provisional application No. 63/384,312, filed on Nov. 18, 2022.

(51) Int. Cl. A61B 1/00; A61B 1/04; A61B 1/267; A61B 34/30

(52) U.S. Cl. CPC A61B 1/00006; A61B 1/00045; A61B 1/043; A61B 1/2676; A61B 34/30; A61B 2034/301

(57) ABSTRACT

Systems and methods for a robotic endoscopy system are provided. The method comprises: (a) receiving instruction to present, at a graphical display, one or both of: one or more tomosynthesis reconstructions or one or more augmented fluoroscopic overlays; and (b) in response to receiving the instruction, causing the graphical display to present one or both of the tomosynthesis reconstructions or the augmented fluoroscopic overlays.

### CROSS-REFERENCE

This application is a continuation of International Application No. PCT/US2023/079481, filed on Nov. 13, 2023, which claims priority to U.S. Provisional Patent Application No. 63/384,312, filed on Nov. 18, 2022, which is entirely incorporated herein by reference.

### BACKGROUND

Early diagnosis of lung cancer is critical. Lung cancer remains the deadliest form of cancer with over 150,000 deaths per year. Compared to computed tomography (CT) guided transthoracic needle aspiration (CT-TTNA), navigational bronchoscopy has a better safety profile (less risk of pneumothorax, life threatening bleeding and length of stay) and the ability to stage the mediastinum but is associated with a lower diagnostic yield. Endoscopy (e.g., bronchoscopy) may involve accessing and visualizing the inside of a patient's lumen (e.g., airways) for diagnostic or therapeutic purposes. During a procedure, a flexible tubular tool such as, for example, an endoscope, may be inserted into the patient's body and an instrument can be passed through the endoscope to a tissue site identified for diagnosis or treatment.

Robotic bronchoscopy systems have gained interest for the biopsy of peripheral lung lesions. Robotic platforms offer superior stability, distal articulation, and visualization over traditional pre-curved catheters. Some of the traditional robotic bronchoscopy systems utilize shape sensing technology (SS) for guidance. SS catheters may have an embedded fiberoptic sensor that measures the shape of the catheter several hundred times a minute. Other traditional robotic bronchoscopy systems incorporate direct visualization, optical pattern recognition and geopositional sensing (OPRGPS) for guidance. Both SS and OPRGPS systems utilize a pre-planning CT scan to create an electronically generated virtual target. However, SS and OPRGPS systems can be prone to CT-to-body divergence (CT2BD). CT2BD is the discrepancy of the electronic virtual target and the actual anatomic location of the peripheral lung lesion. CT2BD can occur for a variety of reasons including atelectasis, neuromuscular weakness due to anesthesia, tissue distortion from the catheter system, bleeding, ferromagnetic interference, and perturbations in anatomy such as pleural effusions. Neither the SS system nor the OPRGPS platform has intra-operative real time correction for CT2BD. In particular, CT2BD can increase the length of the procedure, frustrate the operator, and ultimately result in a nondiagnostic procedure.

### SUMMARY

Digital tomosynthesis algorithms have been recently introduced for the correction of CT2BD. Tomosynthesis (may also be referred to as “tomo”) is limited angle tomography in contrast to full-angle (e.g., 180-degree tomography). However, tomosynthesis reconstruction does not have uniform resolution. For instance, resolution is often the poorest in the depth direction. The standard way to show a 3D volume dataset by three orthogonal planes (e.g., axial, sagittal and coronal) may be ineffective since two of the planes have poorer resolution. A common way to view tomosynthesis volume is to scroll in the depth direction where each slice has good resolution. In the case of pulmonology, it is viewed in the coronal plane and goes through the anterior-posterior (AP) direction by scrolling. Yet this has caused difficulty in determining the spatial relationship of the structures in the depth direction. It can be challenging to determine whether a tool (e.g., biopsy needle) is inside a lesion in the AP direction of a chest tomosynthesis reconstruction.

A need exists for methods and systems capable of determining whether a tool is within a target (e.g., lesion) with improved accuracy or efficiency. The present disclosure addresses the above need by providing a tomosynthesis-based tool-in-lesion decision method with improved accuracy and efficiency. In particular, the provided method may provide a user with quantitative information of the spatial relationship of a thin tool and a target region (e.g., lesion) in the depth direction. The methods, systems, computer-readable media, and techniques herein may identify the positional relationship of the tool and the lesion (in the depth direction) by identifying their depth separately and determine whether the (thin) tool is within the lesion in a quantitative manner.

The method herein may be applied after a robotic platform is set up, target lesions are identified and/or segmented, an airway registration is performed, and an individual target lesion is selected. The method herein may be applied during or after a navigation process to identify a position of a portion of the tool relative to a target. An endoscopy navigation system may use different sensing modalities (e.g., camera imaging data, electromagnetic (EM) position data, robotic position data, etc.). In some cases, the navigation approach may depend on an initial estimate of where the tip of the endoscope is with respect to the airway to begin tracking the tip of the endoscope. Some endoscopy techniques may involve a three-dimensional (3D) model of a patient's anatomy (e.g., CT image), and guide navigation using an EM field and position sensors.

In some cases, a 3D image of a patient's anatomy may be taken one or more times for various purposes. For instance, prior to a medical procedure, a 3D model of a patient anatomy may be created to identify the target location. In some cases, the precise alignment (e.g., registration) between the virtual space of the 3D model, the physical space of the patient's anatomy represented by the 3D model, and the EM field may be unknown. As such, prior to generating a registration, endoscope positions within the patient's anatomy cannot be mapped with precision to corresponding locations within the 3D model. In another instance, during surgical operation, 3D imaging may be performed to update/confirm the location of the target (e.g., lesion) in the case of movement of the target issue or lesion.

In some cases, fluoroscopic imaging systems may be used to determine the location and orientation of medical instruments and patient anatomy within the coordinate system of the surgical environment via fluoroscopy (may also be referred to as “fluoro”). Fluoroscopy is a method providing real-time X-ray imaging. In order for the imaging data to assist in correctly localizing the medical instrument, the coordinate system of the imaging system may be needed for reconstructing the 3D model. For example, multiple 2D fluoroscopy images may be used to create tomosynthesis or Cone Beam CT (CBCT) reconstruction to better visualize and provide 3D coordinates of the anatomical structures. During a CBCT scan, a CBCT scanner may acquire projections along a rotation of 180°-360° angle (i.e. a full rotation of x-ray source and detector) over the region of interest to obtain a volumetric data set. The scanning software collects the data and reconstructs it, producing a digital volume composed of three-dimensional voxels of anatomical data that can then be manipulated and visualized. Tomosynthesis is similar to CBCT scan but uses a limited rotation angle (e.g., 15-60 degrees) thus it has a reduced scanning time as compared to CBCT. Tomosynthesis has an additional benefit over CBCT in that the limited range of motion required for tomosynthesis allows it to be used in more constrained patient settings where full 360° access around the patient is challenging to achieve during a procedure. Tomosynthesis may be performed to determine the location and orientation of medical instruments and patient anatomy. However, traditional tomosynthesis has poor depth resolution (AP direction) causing difficulty in determining whether a tool is within a target region (e.g., lesion) or the position of a thin tool relative to a target region. Systems, methods, and techniques herein beneficially provide tool-in-lesion confirmation in a quantitative manner thereby improving the accuracy and correctness of localizing the tool (e.g., needle) with respect to the target region. As utilized herein, the term CBCT may also refer to tomosynthesis which are utilized interchangeably throughout the specification unless the context suggests otherwise.

As mentioned above, tomosynthesis or CBCT reconstruction of anatomical structures involves combining data from images of 2D projections taken at a plurality of angles with respect to an anatomical structure, and combining the plurality of 2D images to reconstruct a 3D view of the anatomical structure. The mathematical process of combing the 2D projections to create a 3D view requires as an input the relative poses (angles and position) of the camera at which each of the 2D projections is recorded. In some cases, the methods herein may employ pose estimation methods to obtain the relative pose of the camera. For instance, the relative poses of the camera may be obtained by using features within the images themselves. In some examples, when markers (e.g., an array of artificial markers with known positions, or natural features such as bone) are captured within the images, then the relative positions of the markers to one another within the 2D projection may be processed using computer vision methods to estimate the pose of the camera in the 3D world reference frame. In other cases, the pose of the camera at which each of the 2D projections is recorded may be obtained from independent measurements of the camera location and orientation (e.g., accelerometer, IMU, separate imaging device, or other orientation sensors). The present disclosure may utilize the abovementioned methods to generate the construction of 3D views from a combination of 2D projections.

In some cases, features identified from tomosynthesis or CBCT images that are acquired following patient intubation but before commencement of bronchoscopy may be utilized to generate augmented fluoroscopy images. Augmented reality has previously been associated in biopsy with improvements in diagnostic accuracy, procedure time, and radiation dose.

Specifically, augmented fluoroscopy may be utilized for reducing radiation exposure, without compromising diagnostic accuracy. Augmented fluoroscopy may display an augmented layer of information on top of live fluoroscopy view.

In an aspect of the present disclosure, a computer-implemented method is provided for an endoscopic device. The method comprises: (a) providing a first graphical user interface (GUI) for a tomosynthesis mode and a second GUI for a fluoroscopic view mode for viewing a portion of the endoscopic device and a target within a subject; (b) receiving a sequence of fluoroscopic image frames containing the portion of the endoscopic device, a marker, and the target, wherein the sequence of fluoroscopic image frames correspond to various poses of an imaging system acquiring the sequence of fluoroscopic image frames; (c) upon switching to the tomosynthesis mode, i) performing a uniqueness check on the sequence of fluoroscopic image frames and ii) generating a reconstructed 3D tomosynthesis image based at least in part on the poses of the imaging system estimated using the marker; and (d) upon switching to the fluoroscopic view mode, i) generating an estimated pose of the imaging system associated with a fluoroscopic image frame from the sequence of fluoroscopic image frames based at least in part on the marker contained in the fluoroscopic image frame and ii) generating an overlay of the target displayed onto the fluoroscopic image frame based at least in part on the estimated pose.

In a related yet separate aspect, a non-transitory computer-readable media storing instructions is provided. The instructions when executed by at least one processor, cause the at least one processor to perform operations comprising: (a) providing a first graphical user interface (GUI) for a tomosynthesis mode and a second GUI for a fluoroscopic view mode for viewing a portion of the endoscopic device and a target within a subject; (b) receiving a sequence of fluoroscopic image frames containing the portion of the endoscopic device, a marker, and the target, where the sequence of fluoroscopic image frames correspond to various poses of an imaging system acquiring the sequence of fluoroscopic image frames; (c) upon switching to the tomosynthesis mode, i) performing a uniqueness check on the sequence of fluoroscopic image frames and ii) generating a reconstructed 3D tomosynthesis image based at least in part on the poses of the imaging system estimated using the marker; and (d) upon switching to the fluoroscopic view mode, i) generating an estimated pose of the imaging system associated with a fluoroscopic image frame from the sequence of fluoroscopic image frames based at least in part on the marker contained in the fluoroscopic image frame and ii) generating an overlay of the target displayed onto the fluoroscopic image frame based at least in part on the estimated pose.

In some embodiments, the uniqueness check is not performed in the fluoroscopic view mode. In some embodiments, the uniqueness check comprises determining whether a fluoroscopic image frame from the sequence of fluoroscopic image frames is unique based at least in part on an intensity comparison.

In some embodiments, the marker has a 3D pattern. In some cases, the marker comprises a plurality of features placed on at least two different planes. In some embodiments, the marker has a plurality of features of different sizes arranged in a coded pattern. In some cases, the coded pattern comprises a plurality of sub-areas each has a unique pattern. In some cases, in the tomosynthesis mode, the poses of the imaging system are estimated by matching a patch of the plurality of features in the sequence of fluoroscopic image frames to the coded pattern. In some instances, the method further comprises identifying one or more fluoroscopic image frames with high pattern matching scores. In some cases, in the fluoroscopic view mode, the estimated pose of the imaging system is generated by matching a patch of the plurality of features in the fluoroscopic image frame to the coded pattern.

In some embodiments, the first GUI displays the reconstructed 3D tomosynthesis image and is configured to receive a user input on the reconstructed 3D tomosynthesis image indicative of a location of the target. In some cases, the second GUI displays the fluoroscopic image frame with the overlay of the target, and wherein a location of the target displayed on the fluoroscopic image frame is based at least in part on the location of the target.

In some embodiments, a shape of the overlay is based at least in part on a 3D model of the target projected to the fluoroscopic image frame based on the estimated pose. In some cases, the 3D model is generated based on a computed tomography image. In some embodiments, the second GUI provides a graphical element for enabling or disabling a display of the overlay.

In another aspect, systems, methods, and computer-readable media of the present disclosure may implement operations including: (a) in a navigation mode of a graphical user interface (GUI), navigating the endoscopic device towards a target within a subject, the GUI displays a virtual view with visual elements to guide navigating the endoscopic device; (b) upon switching to a tomosynthesis mode of the GUI, i) receiving a sequence of fluoroscopic image frames containing a portion of the endoscopic device and the target, where the sequence of fluoroscopic image frames correspond to various poses of an imaging system acquiring the sequence of fluoroscopic image frames, ii) generating a reconstructed 3D tomosynthesis image based at least in part on the poses of the imaging system and iii) determining a location of the target based at least in part on the reconstructed 3D tomosynthesis image; and (c) upon switching to a fluoroscopic view mode of the GUI, i) obtaining a pose of the imaging system associated with a fluoroscopic image frame acquired in the fluoroscopic view mode, and ii) generating an overlay of the target displayed onto the fluoroscopic image frame based at least in part on the pose of the imaging system and the location of the target determined in (b).

In some embodiments, the virtual view in the navigation mode comprises upon determining a distal tip of the endoscopic device is within a predetermined proximity of the target, rendering a graphical representation of the target and an indicator indicative of an angle of the target relative to an exit axis of a working channel of the endoscopic device. In some embodiments, a location of the target displayed in the navigation mode is updated based on the location of the target determined in (b). In some embodiments, the poses of the imaging system in the tomosynthesis mode are estimated using a marker contained in the sequence of fluoroscopic image frames. In some embodiments, the poses of the imaging system in the tomosynthesis mode are measured by one or more sensors.

In some embodiments, the pose of the imaging system associated with the fluoroscopic image frame in the fluoroscopic view mode is estimated using a marker contained in the fluoroscopic image frame. In some cases, the marker has a 3D pattern. In some instances, the marker comprises a plurality of features placed on at least two different planes. In some cases, the marker has a plurality of features of different sizes arranged in a coded pattern. In some instances, the coded pattern comprises a plurality of sub-areas each has a unique pattern. In some instances, the pose of the imaging system is estimated by matching a patch of the plurality of features in the fluoroscopic image frame to the coded pattern.

In some embodiments, the pose of the imaging system associated with the fluoroscopic image frame in the fluoroscopic view mode is measured by one or more sensors. In some embodiments, in the tomosynthesis mode the sequence of fluoroscopic image frames are processed by performing a uniqueness check on the sequence of fluoroscopic image frames. In some cases, the uniqueness check comprises determining whether a fluoroscopic image frame from the sequence of fluoroscopic image frames is unique based at least in part on an intensity comparison.

In some cases, systems, methods, and computer-readable media of the present disclosure may implement operations including: (a) receiving instruction to present, at one or more graphical displays, one or both of: one or more tomosynthesis reconstructions or one or more augmented fluoroscopic overlays. The tomosynthesis reconstructions may be generated by: (i) acquiring, via a first imaging device of one or more imaging devices, one or more tomosynthesis images over a region of interest of a patient, where at least part of the tomosynthesis images over the region of interest includes first image data corresponding to a plurality of markers and where the tomosynthesis images comprise a plurality of tomosynthesis slices stacked in a depthwise direction, and (ii) generating the tomosynthesis reconstruction based on the tomosynthesis images and the plurality of markers. The tomosynthesis reconstruction includes the tomosynthesis images. The augmented fluoroscopic overlays are generated by: (i) acquiring one or more fluoroscopic images over the region of interest of the patient, where at least part of the fluoroscopic images over the region of interest includes second image data corresponding to the plurality of markers and wherein the fluoroscopic images comprise a plurality of fluoroscopic slices stacked in a depthwise direction, and (ii) generating the augmented fluoroscopic overlay based on the fluoroscopic images and the plurality of markers, where the augmented fluoroscopic overlay includes the augmented fluoroscopic images; and (b) in response to receiving the instruction, causing the one or more graphical displays to present one or both of the tomosynthesis reconstructions or the augmented fluoroscopic overlays.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example process of tomosynthesis image reconstruction.

FIG. 2 shows an example process of augmented fluoroscopy overlay generation.

FIG. 3 shows an example system of various state machines.

FIG. 4 shows an example of a configuration state machine.

FIG. 5 shows example state machine logic.

FIGS. 6A-C shows an example tomosynthesis board marker design.

FIG. 7A shows an example of blob detection of markers on an image of a tomosynthesis board.

FIG. 7B shows an example of candidate points on an image of a tomosynthesis board.

FIG. 7C shows an example of marker extraction on an image of a tomosynthesis board.

FIG. 8 shows an example process for robust tomosynthesis marker matching.

FIG. 9 shows an example result for marker tracking across a tomosynthesis frame sequence on an image of a tomosynthesis board.

FIG. 10 shows an example of a camera pose estimation.

FIG. 11 shows an example of augmented fluoroscopy projection.

FIG. 12 shows examples of robotic bronchoscopy systems, in accordance with some embodiments of the invention.

FIG. 13 shows an example of a fluoroscopy (tomosynthesis) imaging system.

FIG. 14 and FIG. 15 show examples of a flexible endoscope.

FIG. 16 shows an example of an instrument driving mechanism providing mechanical interface to the handle portion of a robotic bronchoscope.

FIG. 17 shows an example of a distal tip of an endoscope.

FIG. 18 shows an example distal portion of the catheter with integrated imaging device and the illumination device.

FIG. 19 shows an example of a user interface comprising a tomosynthesis dashboard.

FIG. 20 shows an example of a user interface comprising a C-arm settings dashboard.

FIG. 21 shows an example of a user interface comprising a scope selection dashboard.

FIG. 22 shows an example of a user interface comprising a selection crosshair panel.

FIG. 23 shows an example of a user interface comprising a lesion selection dashboard.

FIG. 24 shows an example of a user interface comprising an augmented fluoroscopy panel.

FIG. 25 shows an example of a user interface for driving or navigating the endoscope.

FIG. 26 shows an example of the virtual endoluminal view displaying a target.

FIG. 27 shows a computer system that is programmed or otherwise configured to implement methods provided herein.

FIG. 28 shows an example of a method for presenting one or both of tomosynthesis reconstructions or augmented fluoroscopic overlays.

### DETAILED DESCRIPTION

While exemplary embodiments will be primarily directed at tomosynthesis, augmented fluoroscopy, a bronchoscope, etc., one of skill in the art will appreciate that this is not intended to be limiting, and the systems, methods, and techniques described herein may be used for other therapeutic or diagnostic procedures and in other anatomical regions of a patient's body such as a digestive system, including but not limited to the esophagus, liver, stomach, colon, urinary tract, or a respiratory system, including but not limited to the bronchus, the lung, and various others.

The embodiments disclosed herein can be combined in one or more of many ways to provide improved diagnosis and therapy to a patient. The disclosed embodiments can be combined with existing methods and apparatus to provide improved treatment, such as combination with known methods of pulmonary diagnosis, surgery and surgery of other tissues and organs, for example. It is to be understood that any one or more of the structures and steps as described herein can be combined with any one or more additional structures and steps of the methods and apparatus as described herein, the drawings and supporting text provide descriptions in accordance with embodiments.

Although the treatment planning and definition of diagnosis or surgical procedures as described herein are presented in the context of pulmonary diagnosis or surgery, the methods and apparatus as described herein can be used to treat any tissue of the body and any organ and vessel of the body such as brain, heart, lungs, intestines, eyes, skin, kidney, liver, pancreas, stomach, uterus, ovaries, testicles, bladder, ear, nose, mouth, soft tissues such as bone marrow, adipose tissue, muscle, glandular and mucosal tissue, spinal and nerve tissue, cartilage, hard biological tissues such as teeth, bone and the like, as well as body lumens and passages such as the sinuses, ureter, colon, esophagus, lung passages, blood vessels and throat.

As used herein a processor encompasses one or more processors, for example a single processor, or a plurality of processors of a distributed processing system for example. A controller or processor as described herein generally comprises a tangible medium to store instructions to implement steps of a process, and the processor may comprise one or more of a central processing unit, programmable array logic, gate array logic, or a field programmable gate array, for example. In some cases, the one or more processors may be a programmable processor (e.g., a central processing unit (CPU) or a microcontroller), digital signal processors (DSPs), a field programmable gate array (FPGA) or one or more Advanced RISC Machine (ARM) processors. In some cases, the one or more processors may be operatively coupled to a non-transitory computer-readable medium. The non-transitory computer-readable medium can store logic, code, or program instructions executable by the one or more processors unit for performing one or more steps. The non-transitory computer-readable medium can include one or more memory units (e.g., removable media or external storage such as an SD card or random access memory (RAM)). One or more methods or operations disclosed herein can be implemented in hardware components or combinations of hardware and software such as, for example, ASICs, special purpose computers, or general purpose computers.

As used herein, the terms distal and proximal may generally refer to locations referenced from the apparatus and can be opposite of anatomical references. For example, a distal location of a bronchoscope or catheter may correspond to a proximal location of an elongate member of the patient, and a proximal location of the bronchoscope or catheter may correspond to a distal location of the elongate member of the patient.

A system as described herein, includes an elongate portion or elongate member such as a catheter. The terms “elongate member”, “catheter”, “bronchoscope” are used interchangeably throughout the specification unless contexts suggest otherwise. The elongate member can be placed directly into the body lumen or a body cavity. In some embodiments, the system may further include a support apparatus such as a robotic manipulator (e.g., robotic arm) to drive, support, position or control the movements or operation of the elongate member. Alternatively or in addition to, the support apparatus may be a hand-held device or other control devices that may or may not include a robotic system. In some embodiments, the system may further include peripheral devices and subsystems such as imaging systems that would assist or facilitate the navigation of the elongate member to the target site in the body of a subject. Such navigation may require a registration process which will be described later herein.

In some embodiments of the present disclosure, a robotic bronchoscopy system is provided for performing surgical operations or diagnosis with improved performance at low cost. For example, the robotic bronchoscopy system may comprise a steerable catheter that can be entirely disposable. This may beneficially reduce the requirement of sterilization which can be high in cost or difficult to operate, yet the sterilization or sanitization may not be effective. Moreover, one challenge in bronchoscopy is reaching the upper lobe of the lung while navigating through the airways. In some cases, the provided robotic bronchoscopy system may be designed with capability to navigate through the airway having a small bending curvature in an autonomous or semi-autonomous manner. The autonomous or semi-autonomous navigation may require a registration process. Alternatively, the robotic bronchoscopy system may be navigated by an operator through a control system with vision guidance.

A typical lung cancer diagnosis and surgical treatment process can vary drastically, depending on the techniques used by healthcare providers, the clinical protocols, and the clinical sites. The inconsistent processes may cause delay to diagnose lung cancers in early stage, lead to high cost of healthcare system for the patients to diagnose and treat lung cancers, and may cause high risk of clinical and procedural complications. The robotic bronchoscopy system herein may utilize integrated tomosynthesis to improve lesion visibility and tool-in-lesion confirmation, utilize augmented fluoroscopy allowing for real-time navigation updates and guidance in all areas of the lung, thus allowing for standardized early lung cancer diagnosis and treatment.

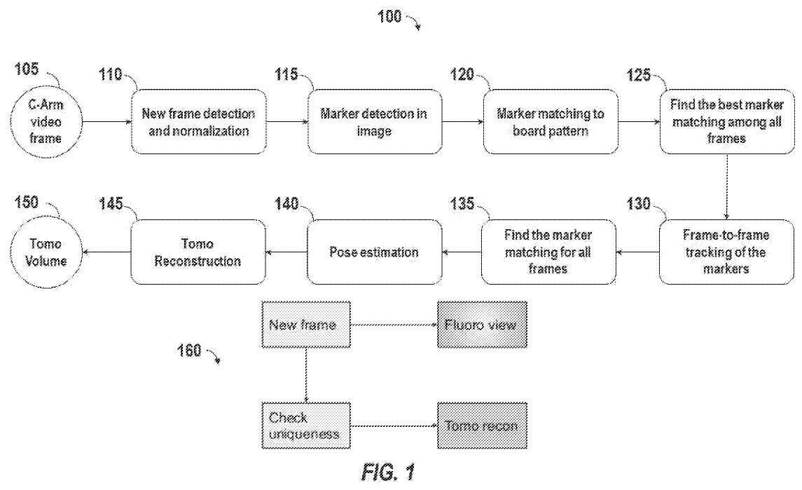

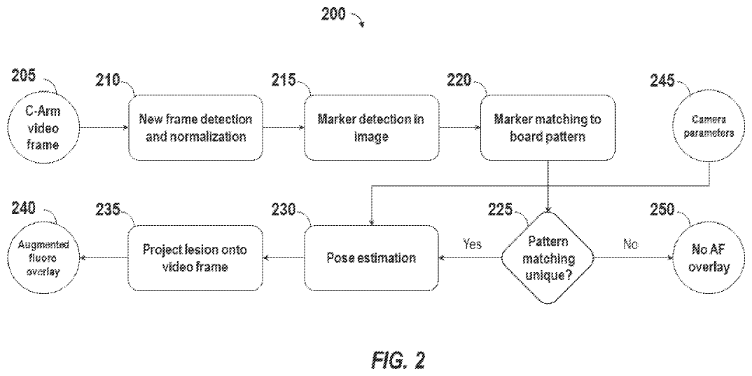

FIG. 1 shows an example process 100 of tomosynthesis image reconstruction. In some cases, the tomosynthesis image reconstruction of the process 100 may comprise generating a 3D volume with a combination of X-ray projection images acquired at different angles (acquired by any type of C-arm systems). FIG. 2 shows an example process 200 of providing augmented fluoroscopy. The augmented fluoroscopy process 200 may comprise projecting a 3D lesion onto the 2D X-ray image as an overlay. The augmented fluoroscopy may display any number of overlays corresponding to multiple lesions or targets. The augmented fluoroscopy may display an overlay for any desired features in addition to a lesion or target. The tomosynthesis imaging mode and the augmented fluoroscopy mode can be accessed from any stage (e.g., during navigation from the driving mode, during performance of operations at the target site, etc.) during an operation session.

Both the process 100 and the process 200 may begin, in some cases, with obtaining C-arm or O-arm video or imaging data using an imaging apparatus such as C-arm imaging system 105, 205, respectively. The C-arm or O-arm imaging system may comprise a source (e.g., an X-ray source) and a detector (e.g., an X-ray detector or X-ray imager). A C-arm imaging system has one or more X-ray sources opposites one or more X-ray detectors and arranged on an arm 1340 that has a “C” shape 1340, where the C-arm may be rotated through some range of angles around a patient. An O-arm is similar to a C-arm but consists of a complete unbroken ring (an “O”) and may be rotated through 360° around a patient. As utilized herein, the term O-arm may be utilized interchangeably throughout the specification with the term C-arm unless the context suggests otherwise.

In some cases, a single C-arm source may provide video or imaging data for the two processes 100 and 200. In some cases, different C-arm sources may provide video or imaging data for the two processes 100 and 200. In some embodiments, the raw video frames may be used for both tomosynthesis and fluoroscopy. However, tomosynthesis may require unique frames from the C-arm, while fluoroscopic view or augmented fluoroscopy may operate using duplicate frames from the C-arm as it is live video, the methods herein may provide a unique frame checking algorithm such that the video frames for tomosynthesis are processed to ensure uniqueness. For example, as illustrated in the process 160, upon receiving a new image frame, if the current mode is tomosynthesis, the image frame may be processed to determine whether it is a unique frame or a duplicate. The uniqueness check may be based on image intensity comparison threshold. For example, a duplicate frame may be identified by comparing the overall average intensity between two frames, or summing over all pixels the absolute difference in intensity between the same pixel in two frames, or summing over the square or other power of the difference in intensity between the same pixel in two frames. For example, when the intensity difference against a previous frame is below a predetermine threshold, the frame may be identified as a duplicate frame and may be removed from being used for tomosynthesis reconstruction. In some cases, the uniqueness or duplicate frame may be identified based on other factors. For instance, the uniqueness check may be based on changes in stochastic noise within the image, even with identical average image intensity. As an example, a frame may be identified as duplicate based on identical average image intensity, but the frame may still be determined as unique if a per pixel comparison shows differences between images. If the current mode is fluoroscopy, the image frame may not be processed for checking uniqueness.

As illustrated, the two processes 100 and 200, may detect the video or imaging frames from the C-arm source at 110 and 210, respectively. In some cases, the video or imaging frames may be normalized. In some cases, normalization may be applied to the image frame to change the range of pixel intensity values in the video or imaging frames. In general, normalization may transform an n-dimension grayscale image I: {X⊆Rⁿ}→{Min, . . . , Max} with intensity values in the range (Min, Max) into a new image I_{NEW}: {X⊆Rⁿ}→{Min_{NEW}, . . . , Max_{NEW}} with intensity values in the range (Min_{NEW}, Max_{NEW}). Examples of possible normalization techniques that may be applied to the C-arm video or image frames in the two processes 100 and 200 (e.g., at 110 or 210), may include linear scaling, clipping, log scaling, z-score, or any other suitable types of normalization.

Accurate camera pose and camera parameters are important for both tomosynthesis image reconstruction and augmented fluoroscopy overlay. The accuracy of marker tracking can affect the pose estimation accuracy or performance. The present disclosure provides an improved method for tracking markers in a sequence of video frames. The method may allow for tomosynthesis reconstruction with improved success rate, allow for larger sweeping angles for tomosynthesis imaging, remove ghosting (due to wrong pose estimation from frame marker mis-tracking) in the 3D reconstructed tomosynthesis image, improve reconstruction quality by using all images and using more uniform angle sampling, and speed up the tomosynthesis reconstruction process.

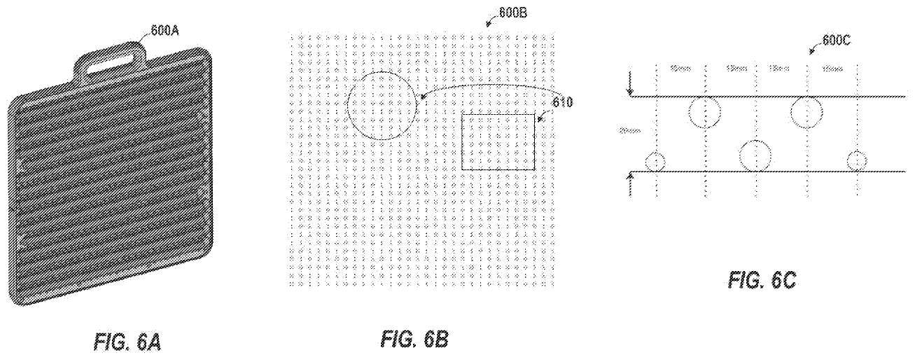

The present disclosure may provide an improved and robust marker tracking methods with improved success rate and higher speed. As shown in the two processes 100 and 200, the same marker detection at 115 and 215, respectively, may be shared in both processes. As will be discussed in further detail in FIGS. 6A-6C, which depict one example of a tomosynthesis board, X-ray projections of markers on a tomosynthesis board may be markers in the X-ray image (obtained via the C-arm, for example). The markers may be detected at 115 and 215 using any suitable image processing techniques. For example, OpenCV's blob detection algorithm may be used to detect markers that are blob-shaped. In some cases, the detected markers (e.g., blobs) may be detected to have certain properties, such as position, shape, size, color, darkness/lightness, opacity, or other suitable properties of markers.

As illustrated, the two processes 100 and 200, may match markers to a board pattern at 120 and 220, respectively. The markers detected at operations 115 and 215 may be matched to the tomosynthesis board (e.g., the tomosynthesis board described with respect to FIG. 6). As described above, the markers may exhibit any number of various physical properties (e.g., position, shape, size, color, darkness/lightness, opacity, etc.) that may be detected at 115 and 215 and may be used for matching the markers to the board pattern at 120 and 220. For example, the tomosynthesis board may have different types of markers such as large blobs and small blobs. In some cases, the large blobs and small blobs may create a pattern which may be used to match the marker pattern in the video or image frames to the pattern on the tomosynthesis board. In some cases, after operations 120 and 220, the processes 100 and 200 may diverge.

As illustrated, after the operation of matching markers to the board pattern 120, the process 100 may find the best marker matching across all video or image frames at 125. The initial marker matching may be the match between markers in the frames and the tomosynthesis board. In some cases, the pattern of the matched markers may be compared over the tomosynthesis board to find the best matching using the Hamming distance. For each frame, the matching with a pattern matching score (e.g., number of matched markers divided by total number of detected markers) may be obtained. The best match may be determined as the match with the highest pattern matching score among all the frames at 125. In some cases, one or more image frames with top pattern matching scores may be identified.

The process 100 may perform frame-to-frame tracking 130. At a high level, the frame-to-frame tracking 130 may include propagating the marker matching from the best match determined at 125 to the rest of the image frames by a robust tomosynthesis marker tracking. In some cases, (i) the markers in a pair of consecutive frames may be initially matched; (ii) each marker in the first frame may then be matched to the k-nearest markers in a second frame; (iii) for each matched pair of markers, a motion displacement between two frames may be computed; (iv) all the markers in the first frame may be transferred to the second frame with the motion displacement; (v) if the motion displacement between a given transferred point from the first frame and a given point location in the second frame is smaller than a threshold, and the two given marker types are the same, then this match may be an inlier; (vi) the best matching may be the motion with the most inliers. From the computed tomosynthesis marker tracking 130, the existing marker matches in the current frame are transferred to the marker matches in the next frame. This process may be repeated for all frames at 135, finding the marker matches for all frames, where the markers in all frames are matched to the tomosynthesis board.

In the augmented fluoroscopy process 200, after matching markers in the video or image frames to the tomosynthesis board at 220, may determine if the pattern matching is unique at 225. The camera pose estimation using markers for augmented fluoroscopy may be more challenging than that for tomosynthesis reconstruction, because (i) only a single video or image frame may be available for augmented fluoroscopy and (ii) the motion information may not be available for removing the ambiguity of the pose estimation. The augmented fluoroscopy algorithm may provide criteria to measure the uniqueness of the matching to the entire tomosynthesis board. In some cases, the marker pattern on the tomosynthesis board may be designed to ensure that the pattern in each sub-area is unique. In some cases, the pattern of the tomosynthesis board may be optimized to maximize the Hamming distances between patches (e.g., any 5×5 patches). In some cases, an in-plane 180-degree rotation may be considered when optimizing the best pattern so that the coincidental alignment is minimized if the board is rotated by 180-degrees either physically or by C-arm setting. Details about the patch/marker matching algorithm and the unique marker design are described later herein.

If the matching is unique, according to the criteria for measuring uniqueness 225, the camera pose may be correctly estimated and the process 200 may advance to pose estimation operation 230. Otherwise, at 225, the augmented fluoroscopy overlay is not displayed and the process 200 advances to operation 250 which may indicate augmented fluoroscopy overlay is available.

Turning to imaging device pose estimation, the processes 100, 200, respectively may recover rotation and translation by minimizing the reprojection error from 3D-2D point correspondences to perform the pose estimation 140, 230. In some cases, Perspective-n-Point (PnP) pose computation may be used to recover the camera poses from n pairs of point correspondences. The minimal form of the PnP problem may be P3P and may be solved with three point correspondences. For each tomosynthesis frame, there may be multiple marker matches, and an estimation method such as RANSAC (Random Sampling with Consensus) variant of PnP solver may be used for pose estimation. In some cases, the pose estimation 140, 230 may be further refined by minimizing the reprojection error using a non-linear minimization method and starting from the initial pose estimate with the PnP solver.

At the tomosynthesis reconstruction 145, the process 100 may perform the tomosynthesis reconstruction based on the pose estimation 140. In some cases, the tomosynthesis reconstruction operation 145 may be implemented as a model in Python (or other suitable programming languages) using the open-source ASTRA (a MATLAB and Python toolbox of high-performance GPU primitives for 2D and 3D tomography) toolbox (or other suitable toolboxes or packages). In the tomosynthesis reconstruction, input to the model may be as follows: (i) undistorted and inpainted (inpainting: a process to restore damaged image) projection images; (ii) estimated projection matrices, such as poses of each projection; and (iii) size, resolution and estimated position of the targeted tomosynthesis reconstruction volume. The output of the model is the tomosynthesis reconstruction (e.g., volume in NifTI format) 145. As such, at operation 150, the process 100 may, in some cases, finish with outputting the tomosynthesis reconstruction for the C-arm systems, where the tomosynthesis reconstruction may include a 3D-volume with a combination of X-ray projection images acquired by the C-arm at various angles.

The operation 235 may comprise using the estimated pose from operation 230 and pre-calibrated camera parameters from operation 245 to project the lesion onto the videoframe. As an example, the lesions may be modeled as ellipsoids that are projected on the 2D fluoroscopic image from the video or image frames as ellipses. It should be noted that the lesion may be modeled using a graphical indicator of any suitable shape, color, transparency, or the like. The augmented fluoroscopy overlay may be displayed on top of the live fluoroscope view corresponding to the lesion projected onto the x-ray image 240. The lesion may be 3D lesion and the 3D lesion is projected to the 2D fluoroscopic image based at least in part on the camera matrix or the pose estimation associated with each 2D fluoroscopic image. Information about the lesion may include 3D location information obtained from the tomosynthesis process. In some cases, shape and size of the lesion may be based on a 3D model of the lesion (created from pre-operation CT or any predetermined parameters). Details about obtaining the lesion information are described elsewhere herein.

### State Machines

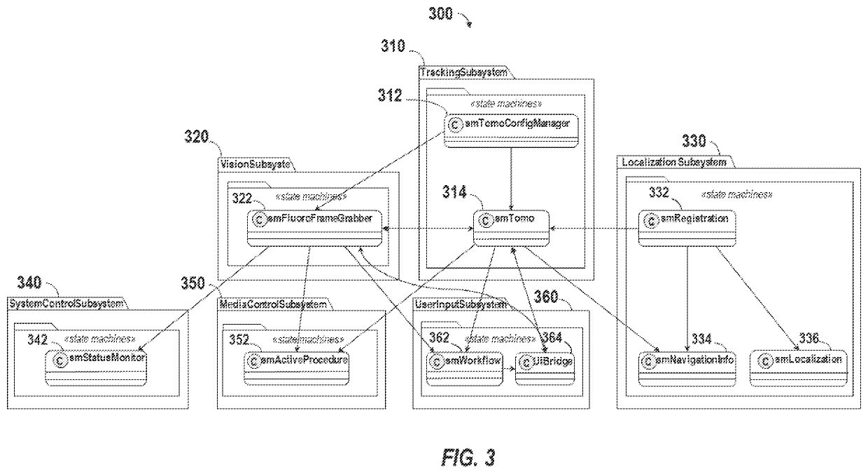

The abovementioned tomosynthesis augmented fluoroscopy overlay methods may be utilized by a tracking system providing a user with real-time location of the lesions, as well as the relative position of the scope or needle and the lesion to correct navigation. FIG. 3 shows an example system 300 of various state machines for implementing a tracking system based at least in part on tomosynthesis and live fluoroscopy with real-time location of the lesions. At a high level, the state machines included in the system 300 may read a set of inputs and change to a different state based on those inputs. The system 300 may include the state a TrackingSubsystem 310, a Vision subsystem 320, a LocalizationSubsystem 330, a SystemControlSubsystem 340, a MediaControlSubsystem 350, and a UserInputSubsystem 360.

In some cases, information for each state machine may comprise functional description of key functionality, system configuration parameters that are owned by the state machine, a state transition diagram, a table that contains details of state transitions, or a table that presents all input and output data of the state machine.

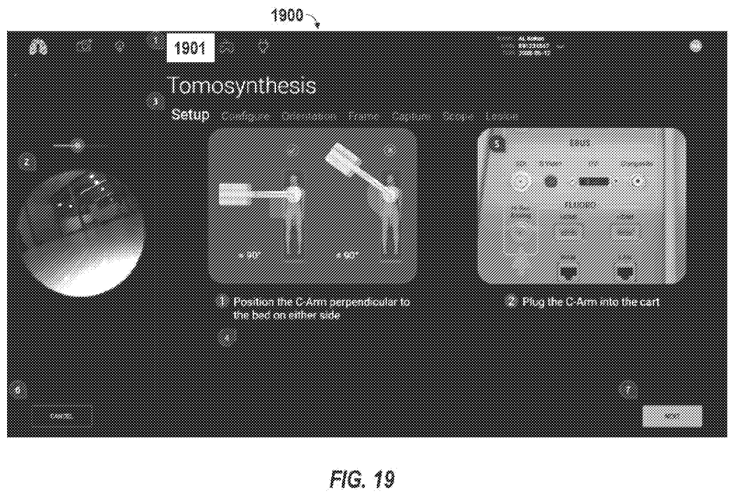

The TrackingSubsystem 310 may comprise two state machines, a smTomoConfigManager 312 and a smTomo 314, as well as helper classes that support the interface between the TrackingSubsystem 310 and other subsystems, software, and hardware components. The TrackingSubsystem 310 may leverage RTI data contracts and implement the described with respect to the smTomoConfigManager 312 and the smTomo 314. The smTomoConfigManager 312 may be responsible for loading tomosynthesis related configuration parameters from configuration files and sending parameters to other state machines through data contracts. In some cases, the configuration parameters have default values (e.g., previous values, recommended values, optimal values, etc.) which can be overwritten by values specified in the configuration files. The smTomo 314 may receive configuration parameters from the smTomoConfigManager 312. The smTomo 314 may retrieve and process fluoroscopy images from smFluoroFrameGrabber 322 of the Vision subsystem 320. The smTomo 314 may receive user commands and may call tomosynthesis dynamic link library (DLL) modules to process and generate intermediate files before tomosynthesis reconstruction. The smTomo 314 may also provide captured unique fluoroscopy images to a treatment interface UI (e.g., as described with respect to FIGS. 19-24) for tip location selection for triangulation calculation to obtain 3D coordinates of a tip. Upon finishing reconstruction, the reconstruction volume may be provided to the treatment interface UI for displaying so that a user can identify and select lesion location coordinates. Tip-to-lesion offset can be obtained and broadcasted to a navigation unit for target driving updates. The smTomo 314 may be responsible for receiving normalized fluoroscopy images, passing to an algorithm, estimating the pose for fluoroscopy images, generating intermediate files, calling a reconstruction module (e.g., a toolbox of 2D and 3D tomography with high-performance GPU speedup) to generate the reconstruction result. The smTomo 314 may perform triangulation calculations to obtain tip coordinates and tip-to-lesion vector calculations based on EM sensor positions and lesion locations. Resulting reconstructions may be displayed in a Treatment UI for the user lesion selection, and lesion information may be broadcasted for augmented fluoroscopy overlay through data contracts.

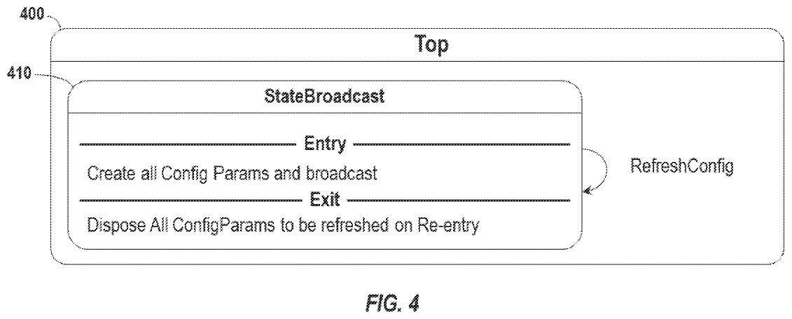

FIG. 4 shows an example of a configuration state machine, a smTomoConfigManager 400 that may be a more detailed view of the smTomoConfigManager 312 of FIG. 3. In some cases, the smTomoConfigManager 400 may read tomosynthesis related configuration parameters. If no entry is found in configuration file for the tomosynthesis related configuration parameters, the smTomoConfigManager 400 may obtain default values (e.g., previous values, recommended values, optimal values, etc.) instead. In some cases, the smTomoConfigManager 400 may broadcast tomosynthesis related configuration parameters through RTI data contracts.

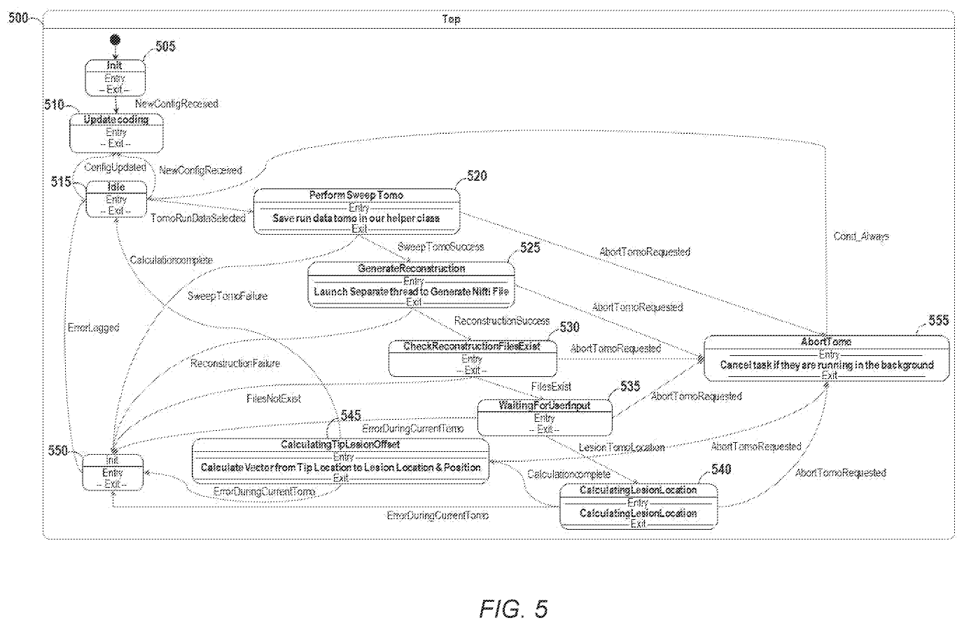

FIG. 5 shows an example of a configuration state machine, a smTomo 500 that may be a more detailed view of the smTomoConfigManager 312 of FIG. 3. The smTomo 314 may receive configuration parameters from the smTomoConfigManager (e.g., the smTomoConfigManager 312 or the smTomoConfigManager 400) at, for example, UpdateConfig module 510. In some cases, the smTomo 500 may receive normalized fluoroscopy image frames from smFluoroFrameGrabber (e.g., the smFluoroFrameGrabber 322). In some cases, the smTomo 500 may generate intermediate files for reconstruction (e.g., tomosynthesis reconstruction) via algorithm modules at, for example, GenerateReconstruction module 525. In some cases, the smTomo 500 may calculate tip coordinates (e.g., via CalculatingTipLesionOffset module 545). In some cases, the smTomo 500 may receive EM sensor data (e.g., from smRegistration 322). Using the EM sensor data, the smTomo 500 may calculate average EM coordinates and obtain a maximum deviation from the average EM coordinates. In some cases, the smTomo 500 may be responsible for pose estimation and generating intermediate images for tomosynthesis reconstruction. If no configuration parameters are found in the configuration file, default values (e.g., general, average, typical, etc.) may be used.

### Marker Board (Tomosynthesis Board)

In some embodiments, the systems herein may provide a marker board (tomosynthesis board) with unique marker design to assist pose estimation with improved efficiency and accuracy. The unique marker design may beneficially allow for a large sweeping angle. Large sweeping angle can beneficially improve reconstruction quality (e.g., improved axial view). FIGS. 6A-6C show an example of a tomosynthesis board 600A with a marker design layout 600B and layering shown in layout 600C. The marker boards described with respect to FIGS. 6A-6C may be applied to one or more of the tomosynthesis or the augmented fluoroscopy techniques also described herein.

The tomosynthesis board 600A may comprise a physical pattern that is unique to transformation or rotation. The physical pattern may be formed of markers in various sizes in predefined code pattern. For example, as illustrated, the tomosynthesis board 600A may comprise dots in different sizes forming a code pattern. In some embodiments, the code pattern may be 3D. In some cases, the dots may be large and small blobs (e.g., beads) that are placed on two layers (with offset in the z-direction of the board as shown in the layout 600C) in a grid pattern according to the marker design layout 600B. In some cases, the offset of the two planes may be sufficient (e.g., offset is at least 20 mm, 30 mm, 40 mm, 50 mm, etc.) such that the 3D pattern of the markers may allow for calibration of the imaging device or pose estimation utilizing a single 2D image of the markers. In some cases, the 3D pattern of the markers may allow for calibration or pose estimation with improved accuracy by utilizing a plurality of 2D images from at projections. In such cases, the offset of the two planes may be small (e.g., no greater than 10 mm, 20 mm, 30 mm, etc.). In some embodiments, the marker board may have a 2D pattern. For example, dots of various sizes may be placed on the same plane.

The blobs may be made of a material visible on an X-ray image, such as metal. The two-layer marker design shown in the side view in the layout 600C of the marker design layout 600B improve accuracy of pose estimation using the tomosynthesis board 600A.

The marker design layout may have a predefined size code pattern. In some cases, the marker design layout 600B may be size coded pattern such that the pattern in each sub-area 610 is unique (“1” represents large bead, “0” represents small bead). A sub-area 610 may be in any shape or size and the pattern within a sub-area is unique. The marker design layout 600B may be optimized to maximize edit distance (e.g., a metric for determining dissimilarity between patterns, strings, etc.) between patches of the tomosynthesis board 600A. In some cases, the edit distance may be measured using the Hamming distances between patches. The patches may be square or rectangular, or some other shape. The patches may be small (e.g., 3×2 patches, 4×6 patches, 5×5 patches, etc.). The patches may be large (e.g., 5×7 patches, 2×9 patches, 9×9 patches, etc.). In some cases, the unique pattern in each sub-area may be designed such that the distance between patches with particular size(s) (e.g., e.g., 3×2 patches, 4×6 patches, 5×5 patches, etc.) may be maximized. Details about the marker matching algorithms are described later herein.

An in-plane rotation (e.g., 90-degree, 180-degree, 270-degree, etc.) rotation may be considered when designing the marker design layout 600B so that the coincidental alignment is minimized if the tomosynthesis board 600A is rotated by rotation, either physically or by C-arm setting. In some cases, a vertical or a horizontal flip may be considered in the marker design layout 600B. A plurality of rows of marker blobs as shown in the side view of the layout 600C may be interlaced in layers (e.g., two layers, three layers, five layers, ten layers, etc.) on the tomosynthesis board 600A.

### Pattern Matching Techniques

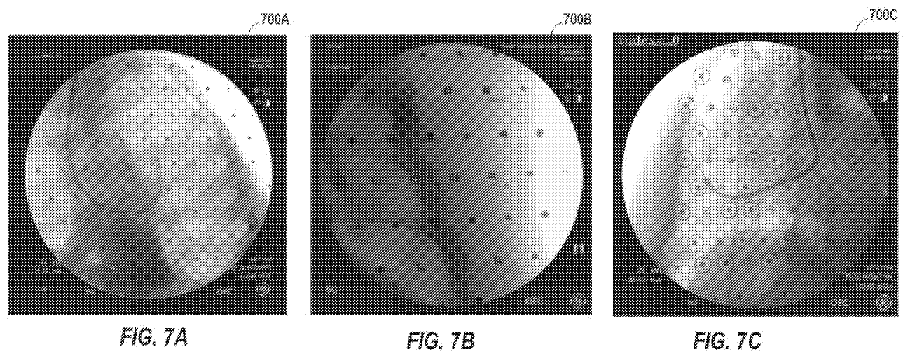

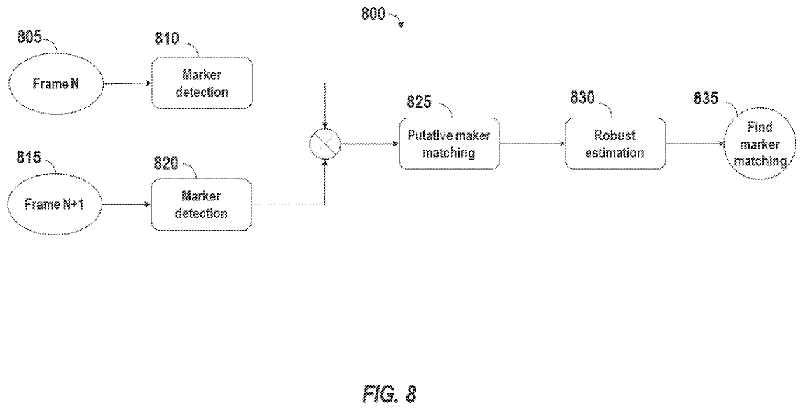

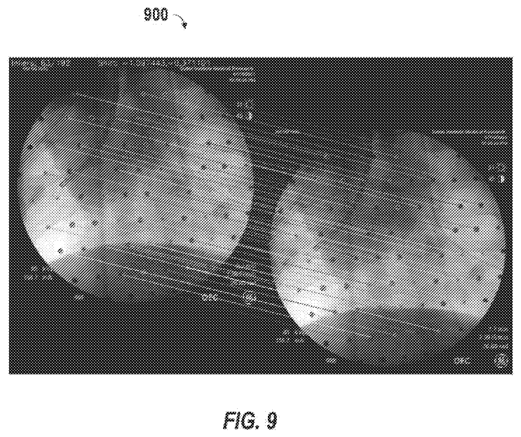

FIGS. 7A-7C illustrate example images used in pattern matching for blob detection. The images and techniques described with respect to 7A-7C, as well as FIGS. 8 and 9, may be applied to one or more of the tomosynthesis or the augmented fluoroscopy techniques also described herein.

FIG. 7A shows an example of blob detection of markers on an image 700A of a tomosynthesis board. The image 700A includes X-ray projections of blobs (e.g., as discussed with respect to FIGS. 6A-6C) on a tomosynthesis board. The blobs are illustrated as markers in the image 700A. The blobs may be detected using any number of image processing techniques, machine learning (e.g., computer vision) techniques, masking techniques, or statistical techniques. For example, the blobs may be detected using any suitable blob detection algorithm. Each detected blob may be marked with various properties, such as center location and radius, as shown in the image 700A. The blobs may be classified into large markers or small markers according to their sizes (e.g., thresholded by the median size of all markers). The large and small markers may create a pattern which is used to match the blob pattern on the tomosynthesis board. While the image 700A, illustrates the markers as blobs, many different patterns, shapes, non-patterns, shading, coloring, etc. could be used (e.g., arrays of various polygons, lines, grid pattern, writings, symbols, etc.). In general, in some cases, the markers may be implemented in various different manners, provided the markers may be useful in matching tomosynthesis images to a spatial position (e.g., with relation to machinery, with relation to the patient, etc.).

FIG. 7B shows an example of candidate points on an image 700B of a tomosynthesis board. The candidate points on the tomosynthesis board grid may be chosen for the initial marker on grid matching. In some cases, a homography model may be used to remove outliers in the initial marker on grid matching. The homography may be computed based on the candidate points between points in an X-ray image (e.g., the images 700A or 700B, etc.) and the tomosynthesis board. For example, estimation techniques, such as RANSAC, may compute the homography based on the candidate points between points in the X-ray image and the tomosynthesis board. The estimation techniques, such as RANSAC, PROSAC (Progressive Sample Consensus), NAPSAC (N Adjacent Points Sample Concensus), etc., may estimate parameters of a mathematical model from a set of observations polluted by outliers. The estimation techniques may repeatedly sample the observations and may reject the outlier samples that do not fit the model and keep the inlier samples that fit the model.

The estimation techniques may implement a model that may be refined with the obtained inlier data via various optimization methods. In some cases, once the homography of one layer of the tomosynthesis board is computed, the rest of the markers on that layer may be extracted provided projections of the blobs are close enough to the markers. The markers left on the image may be fit to the other layer (e.g., the second layer) of the tomosynthesis board.

In some cases, initial marker matching is the match between markers in the image 700B and tomosynthesis board grid. The initial marker matching may be computed over one or more frames of the image 700B. In some cases, the initial match may be a best matched frame (e.g., the frame with the highest matching score among all the frames tested, which may be, in some cases, all the frames in the image 700B). The initial match, with the best matched frame may server as a starting point to propagate the marker matching to the rest of the frames of the image 700B. As such, once the initial marker matching is established, in some cases, then the pattern of the matched markers may “slide” over the image 700B of the tomosynthesis board to find the remaining best matches (e.g., using the Hamming distance). For each frame, a pattern matching score (e.g., number of matched markers divided by total number of detected markers) may obtained; for example, FIG. 7C shows an example of marker extraction on an image 700C depicting pattern matching and computing the pattern matching score. The best matching (e.g., the highest matching score among all the frames) may be chosen as the starting point of pattern matching for all frames of the tomosynthesis sweep.

As illustrated in FIG. 8, which illustrated process 800 for robust tomosynthesis marker matching, once the best matched frame is obtained (e.g., via techniques described with respect to FIGS. 7A-7C), the matching of the best frame may be propagated to all the other frames in tomosynthesis images. In some cases, the process 800 may begin with obtaining a pair of consecutive frames, at 805 and 815, with first markers and second markers, respectively. The process 800 may further include detecting (e.g., via computer vision techniques) at 810 and 820, markers included in the pair of consecutive frames, at 805 and 815, respectively. The process 800 may further include matching (e.g., via k-nearest neighbors), the markers included in the pair of consecutive frames obtained at 805 and 815. The process 800 may further include, for each pair of the matched markers, computing motion displacement between the pair of consecutive frames obtained at 805 and 815. The process 800 may further include, for each of the first markers in the first frame obtained at 805, transferring (e.g., mapping) the first markers to the second frame obtained at 815. The transferring of the first markers to the second markers of the second frame is illustrated in FIG. 9, which depicts an example result for marker tracking across a tomosynthesis frame sequence (of two consecutive frames) on an image of a tomosynthesis board.

Referring again to FIG. 8, for each of the first markers transferred to the second frame, if the distance between the transferred first marker and the corresponding second marker satisfies a threshold (e.g., is less than or equal to a certain distance), then the match between the first marker and the second marker is an inlier at 825. The initial matches may be generated based on distance (e.g., all points within a distance is a match). In some cases, the best matching is the matching with the most inliers. The process 800 may be iterative or repetitive, transferring existing marker matches in a current frame to a next (e.g., consecutive) frame, repeating for all frames at 830, until markers in all frames are matched to the blobs (e.g., beads) on the tomosynthesis board at 835. In some cases, the operation 830 may comprise taking all the above matches and finding the motion that contains the most number of matched marker points and call these matched point pairs inliers.

### Pose Estimation Techniques Using Markers in Images

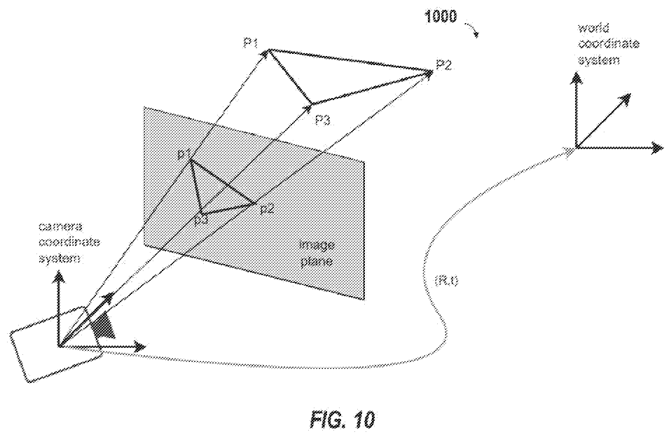

FIG. 10 shows an example diagram 1000 of a camera pose estimation. Reconstructing accurate camera pose and camera parameters may be a key aspect of both tomosynthesis image reconstruction and augmented fluoroscopy overlay. As previously discussed with respect to FIG. 3 and FIG. 5, the smTomo 314 and 500, respectively, may be responsible for estimating poses (e.g., via triangulation) for fluoroscopy images. The pose estimates systems, methods, and techniques described may be applied to one or more of the tomosynthesis or augmented fluoroscopy techniques also described herein.

In the diagram 1000, a pinhole camera model is illustrated. The pinhole camera model of the diagram 1000 may be used to describe the geometry of an X-ray projection. As illustrated in the diagram 1000, pose estimation may include recovering rotation and translation of the camera (camera poses) by minimizing reprojection error from 3D-2D point correspondences. In some cases, an optimization algorithm may be used to refine camera calibration parameters by minimizing the reprojection error. The optimization algorithm may be a least squares algorithm, such as the global Levenberg-Marquardt optimization.

Recovering the camera pose may further include estimating the pose of a calibrated camera given a set of n 3D points in the world and their corresponding 2D projections in the images. The camera pose may include 6 degrees-of-freedom with rotation (e.g., roll, pitch, yaw) and 3D translation of the camera with respect to the world. Perspective-n-Point (PnP) pose computation may be used to recover the camera poses from n pairs of point correspondences. In some cases, n=3, therefore, the minimal form of the PnP problem is P3P, which may be solved with three point correspondences. For each tomosynthesis frame, there may be a plurality of marker matches and a RANSAC or other variant of the PnP solver may be used for the camera pose estimation. Once estimated, the pose may be further refined by minimizing the reprojection error using a non-linear minimization method and starting from the initial pose estimate with the PnP solver.

Performing the camera pose estimation for tomosynthesis reconstruction may include obtaining undistorted images (e.g., from a robotic bronchoscopy system). The undistorted images may have some pre-processing done (e.g., image inpainting, etc.). The undistorted image may be normalized using a normalization algorithm. For example, the undistorted image may be normalized using a logarithmic normalization algorithm, such as Beer's Law: −log log b+δ/(b)+δ, where b is the input image and δ is an offset to avoid a zero logarithm.

### Tomosynthesis Reconstruction Based on Camera Pose

The estimated camera pose or a directly measured camera pose may be utilized in reconstructing the 3D volume image i.e., tomosynthesis reconstruction. In some cases, projection matrices (e.g., estimated camera pose matrices) may be obtained. Additionally, in some cases, physical parameters (e.g., size, resolution, position, volume, geometry, etc.) of the tomosynthesis reconstruction may be obtained. Inputs of one or more of the normalized images, the projection matrices, or the physical parameters, may enable generation of a reconstructed volume for the tomosynthesis reconstruction. To generate the reconstructed volume from the inputs, an algorithm (e.g., PM2vector algorithm) may convert the projection matrices in camera format to vector variables (e.g., in the ASTRA toolbox). Another algorithm may be the same as or similar to the ASTRA FDK Recon algorithm that may call the FDK (Feldkamp, Davis, and Kress) reconstruction module, where normalized projection images may be cosine weighted and ramp filtered, then back-projected to the volume according to the cone-beam geometry. Finally, in some cases, yet another algorithm may convert the reconstructed volume (as output from the ASTRA FDK Recon algorithm, for example) in an appropriate format. For example, a NifTI processing algorithm may save the reconstructed volume as a NifTI image with an affine matrix.

Augmented Fluoroscopy with Camera Pose Estimation

Performing the camera pose estimation for augmented fluoroscopy may allow for achieving a goal of projecting a lesion onto an X-ray image. The present disclosure provides methods for precisely projecting a 3D lesion onto the 2D fluoroscopic image with accurate camera pose and camera parameters. The camera calibration and pose estimation approaches for generating the augmented layer or overlay of the lesion(s) on 2D image can be similar to those described for augmented fluoroscopy. However, the camera pose estimation for augmented fluoroscopy may be, in some cases, more difficult than the pose estimation for tomosynthesis reconstruction because only a single frame is available for the augmented fluoroscopy and motion information may not be available (e.g., for removing the ambiguity of the pose estimation). One or more criteria may be implemented to measure the uniqueness of the matching to the tomosynthesis board. If the matching satisfies the criteria, then the matching may be determined to be unique. Further, when the matching is unique, then the camera pose may be determined to be correctly estimated. The estimated camera pose and pre-calibrated camera parameters may be used to project the 3D lesion onto the fluoroscopic video frame (2D image). If the matching is not unique and the camera pose is not correctly estimated, the augmented fluoroscopy overlay may not be displayed.

In some embodiments, the augmentation layer or the overlay of the target/lesion is displayed over the live fluoroscopic view or the 2D fluoroscopic images in the fluoroscopy mode. The overlay of the target/lesion (e.g., one or more lesions) may be modeled as 3D shapes (e.g., ellipsoids, prisms, spheres, etc.) whose projections on the fluoroscopy image are 2D shapes (e.g., ellipses, polygons, circles, etc.). In some cases, a shape, size or appearance of an overlay of the one or more lesions may be based at least in part on a projection of a lesion 3D models (e.g., 3D meshed model) onto the 2D fluoroscopic images.

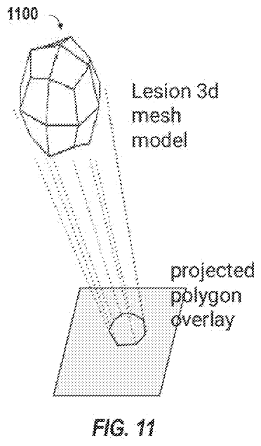

FIG. 11 shows an example of augmented fluoroscopy projection 1100 with a 3D lesion model projected onto a 2D plane (e.g., an image plane), consistent with examples described herein. As shown in the example, the lesion may be modeled as 3D mesh object with multiple corner points. The 3D mesh model may be generated from pre-operation CT or during planning. In the illustrated example, the corner points are projected to the 2D fluoroscopic image where the corner points form a projected polyline contour (from outermost points). Alternatively, the shape or appearance of the overlay for the lesion may be predetermined (e.g., circle, markers, etc.) that may not be based on a 3D meshed model from imaging.

In some cases, the location of the overlay may be determined based at least in part on the target/lesion location determined from the tomosynthesis or the reconstructed 3D tomosynthesis images and a pose estimation associated with the 2D fluoroscopic image.

Pose Estimation Techniques without Using Markers

Relative camera poses at which images are acquired are required inputs for tomosynthesis reconstruction of 3D volumes and also for augmented fluoroscopy. Methods and systems for accurately determining the relative camera poses at which images are acquired can be utilized to provide the pose inputs required for tomosynthesis and augmented fluoroscopy. In some embodiments, the camera pose may be obtained without markers. In some cases, methods herein may obtain camera poses without utilizing makers which beneficially allow for higher quality images to be achieved as markers may partially obscure the images. For example, in tomosynthesis mode, a higher quality 3D reconstruction of the volume may be achieved without markers' presence in the images, since prior to performing tomosynthesis the region of an image around each marker is typically excised from the image, reducing the overall amount of information available with which to generate the 3D reconstruction of the volume.

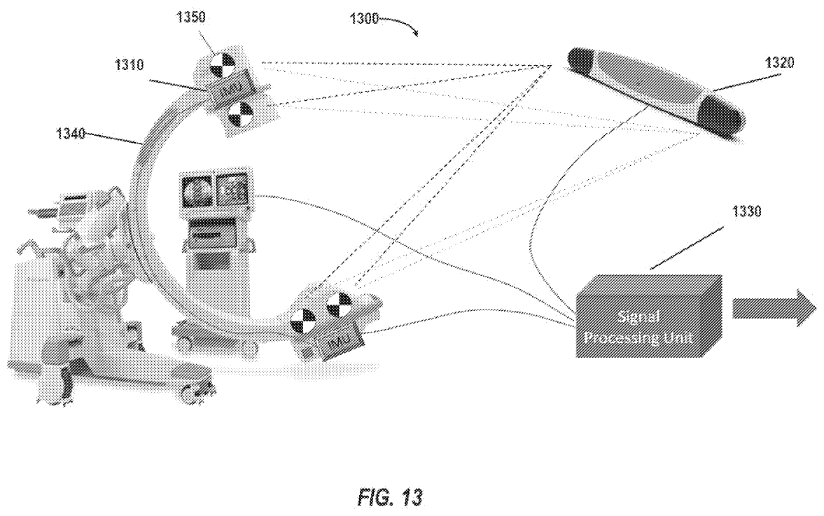

As illustrated in FIG. 13, the pose or motion of the fluoroscopy (tomosynthesis) imaging system may be measured directly using any suitable motion/location sensors 1310 disposed on the fluoroscopy (tomosynthesis) imaging system. The motion/location sensors may include, for example, inertial measurement units (IMUs)), one or more gyroscopes, velocity sensors, accelerometers, magnetometers, location sensors (e.g., global positioning system (GPS) sensors), vision sensors (e.g., imaging devices capable of detecting visible, infrared, or ultraviolet light, such as cameras), proximity or range sensors (e.g., ultrasonic sensors, lidar, time-of-flight or depth cameras), altitude sensors, attitude sensors (e.g., compasses) or field sensors (e.g., magnetometers, electromagnetic sensors, radio sensors). In some cases, the fluoroscopy system may comprise rotary or linear encoders or similar means of measuring rotational motion of the arm with respect to the structure supporting and holding the arm in position. The encoders may also be used to provide pose of the imaging devices. In some cases, the one or more sensors for tracking the motion and location of the fluoroscopy (tomosynthesis) imaging station may be disposed on the imaging station or be located remotely from the imaging station, such as a wall-mounted camera 1320. The various poses may be captured by the one or more sensors as described above.

In some cases, when the source and detector relative poses are known from motion/location sensors, makers (e.g., a pattern of blobs or beads within a frame to estimate pose) may not be required to estimate the camera pose. In some cases, when a pose information is available from multiple sources, such as both from a direct pose measurement (e.g., motion/location sensors) and pose estimation (e.g., image analysis of features within a frame), the pose information (e.g., direct measurement and estimated pose) from the multiple sources may be combined to provide a more accurate pose estimation. For example, the direct pose measurement and the estimated pose based on computer vision may be averaged (or weighted) to generate a final pose for the imaging system.

In some cases, the C-arm imaging system undergoes only rotations around an axis of rotation with no overall translations, for tomosynthesis reconstruction or augmented fluoroscopy the pose information required for each image may include only the relative angles between the images. The relative angles between images may be measured by many of the methods described above. For example, a 3D accelerometer may be mounted to the C-arm and the direction of the acceleration due to Earth's gravity may be used to determine relative changes of the angle of the camera as the C-arm is rotated. For the case where the C-arm may be both rotating and translating, the complete 6 degrees of freedom (6DOF) of the camera may need to be known as inputs into tomography or augmented fluoroscopy. For this case, for example, a binocular optical “localizer” system 1320 along with localizer fiducial markers 1350 mounted to the C-arm 1340 may provide the complete 6DOF information for the (x, y, z) location and (Rx, Ry, Rz) orientation of the frame of the fiducial markers. A (one time) camera calibration process may be performed to know the translation and rotation transformations from the localizer fiducial marker frame to the camera frame. After calibration, the 6DOF pose of the camera may be known at the time each image is acquired based on captured data from the localizer.

### Robotic Bronchoscopy System

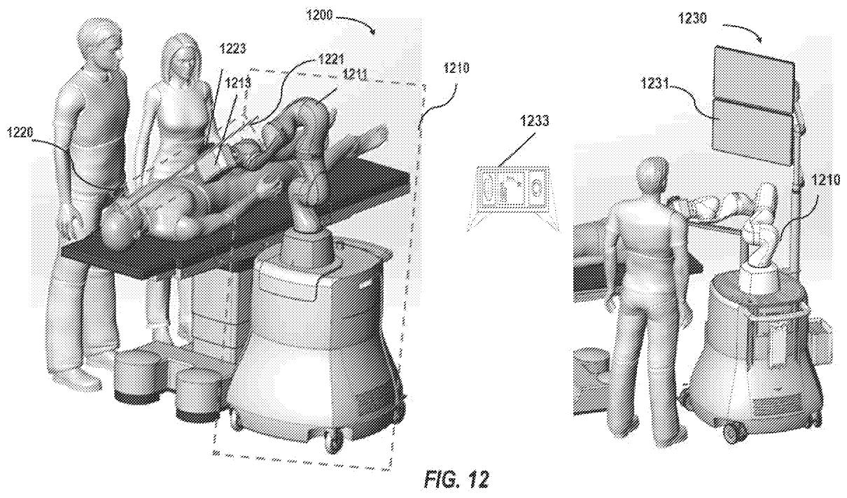

FIG. 12 show examples of robotic bronchoscopy system 1200, 1230, in accordance with some examples. The robotic bronchoscopy system may implement the methods, subsystems and functional modules as described above. As shown in FIG. 12, the robotic bronchoscopy system 1200 may comprise a steerable catheter assembly 1220 and a robotic support system 1210, for supporting or carrying the steerable catheter assembly. The steerable catheter assembly can be a bronchoscope. In some embodiments, the steerable catheter assembly may be a single-use robotic bronchoscope. In some embodiments, the robotic bronchoscopy system 1200 may comprise an instrument driving mechanism 1213 that is attached to the arm of the robotic support system. The instrument driving mechanism may be provided by any suitable controller device (e.g., hand-held controller) that may or may not include a robotic system. The instrument driving mechanism may provide mechanical and electrical interface to the steerable catheter assembly 1220. The mechanical interface may allow the steerable catheter assembly 1220 to be releasably coupled to the instrument driving mechanism. For instance, a handle portion of the steerable catheter assembly can be attached to the instrument driving mechanism via quick install/release means, such as magnets, spring-loaded levels and the like. In some cases, the steerable catheter assembly may be coupled to or released from the instrument driving mechanism manually without using a tool.

The steerable catheter assembly 1220 may comprise a handle portion 1223 that may include components configured to processing image data, provide power, or establish communication with other external devices. For instance, the handle portion 1223 may include a circuitry and communication elements that enables electrical communication between the steerable catheter assembly 1220 and the instrument driving mechanism 1213, and any other external system or devices. In another example, the handle portion 1223 may comprise circuitry elements such as power sources for powering the electronics (e.g., camera and LED lights) of the endoscope. In some cases, the handle portion may be in electrical communication with the instrument driving mechanism 1213 via an electrical interface (e.g., printed circuit board) so that image/video data or sensor data can be received by the communication module of the instrument driving mechanism and may be transmitted to other external devices/systems. Alternatively or in addition to, the instrument driving mechanism 1213 may provide a mechanical interface only. The handle portion may be in electrical communication with a modular wireless communication device or any other user device (e.g., portable/hand-held device or controller) for transmitting sensor data or receiving control signals. Details about the handle portion are described later herein.

The steerable catheter assembly 1220 may comprise a flexible elongate member 1211 that is coupled to the handle portion. In some embodiments, the flexible elongate member may comprise a shaft, steerable tip and a steerable section. The steerable catheter assembly may be a single use robotic bronchoscope. In some cases, only the elongate member may be disposable. In some cases, at least a portion of the elongate member (e.g., shaft, steerable tip, etc.) may be disposable. In some cases, the entire steerable catheter assembly 1220 including the handle portion and the elongate member can be disposable. The flexible elongate member and the handle portion are designed such that the entire steerable catheter assembly can be disposed of at low cost. Details about the flexible elongate member and the steerable catheter assembly are described later herein.

In some embodiments, the provided bronchoscope system may also comprise a user interface. As illustrated in the example system 1230, the bronchoscope system may include a treatment interface module 1231 (user console side) or a treatment control module 1233 (patient and robot side). The treatment interface module may allow an operator or user to interact with the bronchoscope during surgical procedures. In some embodiments, the treatment control module 1233 may be a hand-held controller. The treatment control module may, in some cases, comprise a proprietary user input device and one or more add-on elements removably coupled to an existing user device to improve user input experience. For instance, physical trackball or roller can replace or supplement the function of at least one of the virtual graphical element (e.g., navigational arrow displayed on touchpad) displayed on a graphical user interface (GUI) by giving it similar functionality to the graphical element which it replaces. Examples of user devices may include, but are not limited to, mobile devices, smartphones/cellphones, tablets, personal digital assistants (PDAs), laptop or notebook computers, desktop computers, media content players, and the like. Details about the user interface device and user console are described later herein.

The user console 1231 may be mounted to the robotic support system 1210. Alternatively or in addition to, the user console or a portion of the user console (e.g., treatment interface module) may be mounted to a separate mobile cart.

The present disclosure provides a robotic endoluminal platform with integrated tool-in-lesion tomosynthesis technology. In some cases, the robotic endoluminal platform may be a bronchoscopy platform. The platform may be configured to perform one or more operations consistent with the method described herein. FIG. 13 shows an example of a robotic endoluminal platform and its components or subsystems, in accordance with some embodiments of the invention. In some embodiments, the platform may comprise a robotic bronchoscopy system and one or more subsystems that can be used in combination with the robotic bronchoscopy system of the present disclosure.

In some embodiments, the one or more subsystems may include imaging systems such as a fluoroscopy imaging system for providing real-time imaging of a target site (e.g., comprising lesion). Multiple 2D fluoroscopy images may be used to create tomosynthesis or Cone Beam CT (CBCT) reconstruction to better visualize and provide 3D coordinates of the anatomical structures. FIG. 13 shows an example of a fluoroscopy (tomosynthesis) imaging system 1300. For example, the fluoroscopy (tomosynthesis) imaging system may perform accurate lesion location tracking or tool-in-lesion confirmation before or during surgical procedure as described above. In some cases, lesion location may be tracked based on location data about the fluoroscopy (tomosynthesis) imaging system/station (e.g., C arm) and image data captured by the fluoroscopy (tomosynthesis) imaging system. The lesion location may be registered with the coordinate frame of the robotic bronchoscopy system.

In some cases, a location, pose or motion of the fluoroscopy imaging system may be measured/estimated to register the coordinate frame of the image to the robotic bronchoscopy system, or for constructing the 3D model/image. In some cases, the pose of the imaging system may be estimated using the pose estimation methods as described elsewhere herein. For example, pose estimation method based on the unique marker boards may be employed to obtain the imaging device pose associated with each 2D image.

Alternatively, the pose or motion of the fluoroscopy (tomosynthesis) imaging system may be measured directly using any suitable motion/location sensors 1310 disposed on the fluoroscopy (tomosynthesis) imaging system. The motion/location sensors may include, for example, inertial measurement units (IMUs)), one or more gyroscopes, velocity sensors, accelerometers, magnetometers, location sensors (e.g., global positioning system (GPS) sensors), vision sensors (e.g., imaging devices capable of detecting visible, infrared, or ultraviolet light, such as cameras), proximity or range sensors (e.g., ultrasonic sensors, lidar, time-of-flight or depth cameras), altitude sensors, attitude sensors (e.g., compasses) or field sensors (e.g., magnetometers, electromagnetic sensors, radio sensors). In some cases, the fluoroscopy system may comprise rotary or linear encoders or similar means of measuring rotational motion of the arm with respect to the structure supporting and holding the arm in position. The encoders may also be used to provide pose of the imaging devices. In some cases, the one or more sensors for tracking the motion and location of the fluoroscopy (tomosynthesis) imaging station may be disposed on the imaging station or be located remotely from the imaging station, such as a wall-mounted camera 1320. The various poses may be captured by the one or more sensors as described above. For the case where the source and detector relative poses are known from motion/location sensors it is not required to use a pattern of blobs or beads within a frame to estimate pose. In some cases, when a pose information is available from multiple sources, such as both from a direct pose measurement (e.g., motion/location sensors) and pose estimation (e.g., image analysis of features within a frame), the pose information (e.g., direct measurement and estimated pose) from the multiple sources may be combined to provide a more accurate pose estimation. For example, the direct pose measurement and the estimated pose based on computer vision may be averaged (or weighted) to generate a final pose for the imaging system.

In some embodiments, a location of a lesion may be segmented in the image data captured by the fluoroscopy (tomosynthesis) imaging system with aid of a signal processing unit 1330. One or more processors of the signal processing unit may be configured to further overlay treatment locations (e.g., lesion) on the real-time fluoroscopic image/video. For example, the processing unit may be configured to generate an augmented layer comprising augmented information such as the location of the treatment location or target site. In some cases, the augmented layer may also comprise a graphical marker indicating a path to this target site. The augmented layer may be a substantially transparent image layer comprising one or more graphical elements (e.g., box, arrow, etc.). The augmented layer may be superposed onto the optical view of the optical images or video stream captured by the fluoroscopy (tomosynthesis) imaging system, or displayed on the display device. The transparency of the augmented layer allows the optical image to be viewed by a user with graphical elements overlay on top of the optical image. In some cases, both the segmented lesion images and an optimum path for navigation of the elongate member to reach the lesion may be overlaid onto the real time tomosynthesis images. This may allow operators or users to visualize the accurate location of the lesion as well as a planned path of the bronchoscope movement. In some cases, the segmented and reconstructed images (e.g., CT images as described elsewhere) provided prior to the operation of the systems described herein may be overlaid on the real time images.

In some embodiments, the one or more subsystems of the platform may comprise one or more treatment subsystems such as manual or robotic instruments (e.g., biopsy needles, biopsy forceps, biopsy brushes) or manual or robotic therapeutical instruments (e.g., RF ablation instrument, Cryo instrument, Microwave instrument, and the like).

In some embodiments, the one or more subsystems of the platform may comprise a navigation and localization subsystem. The navigation and localization subsystem may be configured to construct a virtual airway model based on the pre-operative image (e.g., pre-op CT image or tomosynthesis). The navigation and localization subsystem may be configured to identify the segmented lesion location in the 3D rendered airway model and based on the location of the lesion, the navigation and localization subsystem may generate an optimal path from the main bronchi to the lesions with a recommended approaching angle towards the lesion for performing surgical procedures (e.g., biopsy).

In some cases, to assist with reaching the target tissue location, the location and movement of the medical instruments may be registered with intra-operative 3D images of the patient anatomy. In some cases, this may be accomplished by determining the transformation from the reference frame of the 3D images to the reference frame of the EM field or other navigation solution, allowing the location of the lesion within the 3D model of the patient anatomy to be updated based on data from the intra-operative 3D images of the patient anatomy. The transformation between the reference frame of the 3D image and the reference frame of the EM or other navigation system (co-registration) may comprise the three rotations between the frames and the three translations between the frames.

The present disclosure may provide co-registration methods to co-register the reference frame of the 3D images and the navigation reference frame (e.g., reference frame of the EM field). In some cases, the co-registration method may utilize markers visible within the image dataset to establish the reference frame for the 3D image. The marker reference frame and the EM reference frame (or other navigation reference frame) may have known transformation relationship (e.g., rotations and translations between the marker reference frame and the EM reference frame are known during equipment setup or from device mechanical constraints). The location of the patient anatomy is found within the 3D image reference frame, and from mechanical construction or setup a transformation from the 3D image reference frame to the navigation reference frame is obtained, and the position of the patient anatomy within the navigation reference frame may be updated based upon the measured patient location within the 3D image.

In some cases, instead of obtaining the rotations and translations between the marker reference frame and the navigation reference frame (e.g., EM reference frame) from equipment setup or from device mechanical constraints, only the rotations of the marker reference frame with respect to an navigation reference frame (e.g., with both a marker frame and an EM generator affixed to a bed with the (x,y,z) axes of the frames parallel to the principal axes of the bed) are obtained from the equipment setup or from device mechanical constraints, whereas the translations between the frames are obtained based on real-time measurements. For example, the translations relationship may be obtained by measuring the (x, y, z) positions of a features/structure (e.g., tip, any fiducial marker, part of the endoscope, etc.) in both the navigation and the imaging systems. For instance, with EM navigation, the (x, y, z) position of the tip of an endoscope is measured in the frame of the EM navigation system. The (x, y, z) position of that tip of the endoscope in the 3D image reference frame is measured by locating the tip within the 3D data set containing the tip concurrently with the EM measurement. It should be noted any structure/feature (e.g., endoscope tip, tool, marker on the tool, etc.) with a position that can be measured in both the navigation system and the imaging system can be utilized to determine the translation relationship between the two frames.

In some cases, both the rotation and translation relationship between the two frame can be obtained by using a structure or feature which can be located in (x,y,z) by both the marker reference frame and the navigation reference frame. For example, with EM navigation, both the (x,y,z) position and the (Rx, Ry, Rz) angular orientation of the tip of an endoscope in the frame of the EM navigation system is measured. Structures or features can be constructed into an endoscope that are opaque to x-rays and that allow the position and angular orientation of the structure to be determined by the 3D reconstruction. Based on the endoscope tip (x, y, z) and (Rx, Ry, Rz) determined in both the 3D image frame and the EM frame, the two reference frames can be co-registered.

In some cases, a radio opaque marker affixed to the EM (or other navigation system) may be utilized to obtain the transformation matrix. For instance, by construction or calibration, the markers affixed to the EM system may have known translations and orientations with respect to the EM frame. The markers on the EM frame may be visible in the 3D images which can be used to determine the translations and orientations of the markers affixed to the EM system with respect to the markers in the 3D image. Next, the method may combine the transformations to determine the position of the physiology (e.g., a lesion) within the EM frame as EMframe_T_lesion=EMframe_T_EMmarkers*EMmarkers_T_3Dframe*3Dframe_T_lesion, where each Frame2_T_Frame1 label means the 4×4 rotation and translation transformation matrix that provides the (x, y, z) position of a point in the Frame 2 coordinate system given the (x, y, z) position of the same point in the Frame 1 coordinate system.

In some cases, the co-registration method may comprise independently measuring the relative position and orientation of the c-arm camera with respect to the navigation reference frame, e.g., using one set of 3D localization tools 1310 or 1350 affixed to structures 1340 in physical connection to and with known orientation to the camera, and a second set of 3D localization tools affixed to structures in physical connection to and with known orientation to the EM frame.

In some cases, the co-register method may include structures or features to the C-arm that allow the EM navigation (or other navigation system) to measure the translations and angles of the camera with respect to the EM frame. For example, a 6-DOF EM sensor may be affixed to the C-arm such that the position and orientation of the sensor can be measured by the EM navigation system in the EM frame. The position and orientation of the EM sensor with respect to the camera may be known from construction or calibration, and so measurement of the position and orientation of the EM sensor provides the position and orientation of the camera in the EM frame. The 3D image may be reconstructed from multiple 2D projection using the individual camera poses as described elsewhere herein, and as the camera pose is already in the EM frame, the reconstructed 3D image frame is automatically co-registered to the EM reference frame (they are the same frame). This method beneficially eliminates the requirement of using computer vision or features in image for co-registration.

With the image-guided instruments registered to the images, the instruments may navigate natural or surgically created passageways in anatomical systems such as the lungs, the colon, the intestines, the kidneys, the heart, the circulatory system, or the like. In some instances, after the medical instrument (e.g., needle, endoscope) reaches the target location or after a surgical operation is completed, 3D imaging may be performed to confirm the instrument or operation is at the target location.

At a registration step before driving the bronchoscope to the target site, the system may align the rendered virtual view of the airways to the patient airways. Image registration may consist of a single registration step or a combination of a single registration step and real-time sensory updates to registration information. The registration process may include finding a transformation that aligns an object (e.g., airway model, anatomical site) between different coordinate systems (e.g., EM sensor coordinates, and patient 3D model coordinates based on pre-operative CT imaging). Details about the registration are described later herein.

Once registered, all airways may be aligned to the pre-operative rendered airways. During robotic bronchoscope driving towards the target site, the location of the bronchoscope inside the airways may be tracked and displayed. In some cases, location of the bronchoscope with respect to the airways may be tracked using positioning sensors. Other types of sensors (e.g., camera) can also be used instead of or in conjunction with the positioning sensors using sensor fusion techniques. Positioning sensors such as electromagnetic (EM) sensors may be embedded at the distal tip of the catheter and an EM field generator may be positioned next to the patient torso during procedure. The EM field generator may locate the EM sensor position in 3D space or may locate the EM sensor position and orientation with 5 or 6 degrees of freedom (5DOF or 6DOF), consisting of the 3 spatial coordinates and 2 or 3 orientation angles. This may provide a visual guide to an operator when driving the bronchoscope towards the target site.

In real-time EM tracking, the EM sensor comprising of one or more sensor coils embedded in one or more locations and orientations in the medical instrument (e.g., tip of the endoscopic tool) measures the variation in the EM field created by one or more static EM field generators positioned at a location close to a patient. The location information detected by the EM sensors is stored as EM data. The EM field generator (or transmitter) may be placed close to the patient to create a low intensity low frequency alternating magnetic field that the embedded sensor may detect. The alternating magnetic field induces small currents in the sensor coils of the EM sensor, which may be analyzed to determine the distance and angle between the EM sensor and the EM field generator. These distances and orientations may be intra-operatively registered to the patient anatomy (e.g., 3D model) to determine the registration transformation that aligns a single location in the coordinate system with a position in the pre-operative model of the patient's anatomy.

In some embodiments, the platform herein may utilize fluoroscopic imaging systems to determine the location and orientation of medical instruments and patient anatomy within the coordinate system of the surgical environment. In particular, the systems and methods herein may employ a mobile C-arm fluoroscopy as a low-cost and mobile real-time qualitative assessment tool. Fluoroscopy is an imaging modality that obtains real-time moving images of patient anatomy, and medical instruments. Fluoroscopic systems may include C-arm systems which provide positional flexibility and are capable of orbital, horizontal, or vertical movement via manual or automated control. Fluoroscopic image data from multiple viewpoints (i.e., with the fluoroscopic imager moved among multiple locations) in the surgical environment may be compiled to generate two-dimensional or three-dimensional tomographic images. When using a fluoroscopic imager system that includes a digital detector (e.g., a flat panel detector), the generated and compiled fluoroscopic image data may permit the sectioning of planar images in parallel planes according to tomosynthesis imaging techniques. The C-arm imaging system may comprise a source (e.g., an X-ray source) and a detector (e.g., an X-ray detector or X-ray imager). The X-ray detector may generate an image representing the intensities of received x-rays. The imaging system may reconstruct 3D image based on multiple 2D image acquired from a wide range of angels. In some cases, the rotation angle range may be at least 120-degree, 130-degree, 140-degree, 150-degree, 160-degree, 170-degree, 180-degree or greater. In some cases, the 3D image may be generated based on a pose of the X-ray imager.

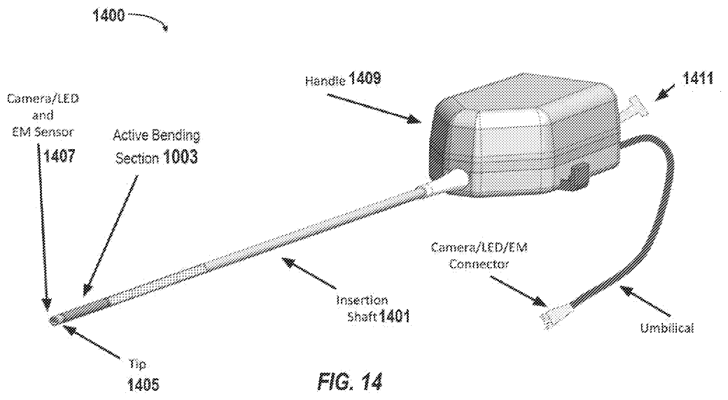

The bronchoscope or the catheter may be disposable. FIG. 14 illustrates an example of a flexible endoscope 1400, in accordance with some embodiments of the present disclosure. As shown in FIG. 14, the flexible endoscope 1400 may comprise a handle/proximal portion 1409 and a flexible elongate member to be inserted inside of a subject. The flexible elongate member can be the same as the one described above. In some embodiments, the flexible elongate member may comprise a proximal shaft (e.g., insertion shaft 1401), steerable tip (e.g., tip 1405), and a steerable section (active bending section 1403). The active bending section, and the proximal shaft section can be the same as those described elsewhere herein. The endoscope 1400 may also be referred to as steerable catheter assembly as described elsewhere herein. In some cases, the endoscope 1400 may be a single-use robotic endoscope. In some cases, the entire catheter assembly may be disposable. In some cases, at least a portion of the catheter assembly may be disposable. In some cases, the entire endoscope may be released from an instrument driving mechanism and can be disposed of. In some embodiments, the endoscope may contain varying levels of stiffness along the shaft, as to improve functional operation.

The endoscope or steerable catheter assembly 1400 may comprise a handle portion 1409 that may include one or more components configured to process image data, provide power, or establish communication with other external devices. For instance, the handle portion may include circuitry and communication elements that enable electrical communication between the steerable catheter assembly 1400 and an instrument driving mechanism (not shown), and any other external system or devices. In another example, the handle portion 1409 may comprise circuitry elements such as power sources for powering the electronics (e.g., camera, electromagnetic sensor, and LED lights) of the endoscope.

The one or more components located at the handle may be optimized such that expensive and complicated components may be allocated to the robotic support system, a hand-held controller or an instrument driving mechanism thereby reducing the cost and simplifying the design of the disposable endoscope. The handle portion or proximal portion may provide an electrical and mechanical interface to allow for electrical communication and mechanical communication with the instrument driving mechanism. The instrument driving mechanism may comprise a set of motors that are actuated to rotationally drive a set of pull wires of the catheter. The handle portion of the catheter assembly may be mounted onto the instrument drive mechanism so that its pulley/capstans assemblies are driven by the set of motors. The number of pulleys may vary based on the pull wire configurations. In some cases, one, two, three, four, or more pull wires may be utilized for articulating the flexible endoscope or catheter.

The handle portion may be designed allowing the robotic bronchoscope to be disposable at reduced cost. For instance, classic manual and robotic bronchoscopes may have a cable in the proximal end of the bronchoscope handle. The cable often includes illumination fibers, camera video cable, and other sensors fibers or cables such as electromagnetic (EM) sensors, or shape sensing fibers. Such complex cables can be expensive adding to the cost of the bronchoscope. The provided robotic bronchoscope may have an optimized design such that simplified structures and components can be employed while preserving the mechanical and electrical functionalities. In some cases, the handle portion of the robotic bronchoscope may employ a cable-free design while providing a mechanical/electrical interface to the catheter.

The electrical interface (e.g., printed circuit board) may allow image/video data or sensor data to be received by the communication module of the instrument driving mechanism and may be transmitted to other external devices/systems. In some cases, the electrical interface may establish electrical communication without cables or wires. For example, the interface may comprise pins soldered onto an electronics board such as a printed circuit board (PCB). For instance, receptacle connector (e.g., the female connector) is provided on the instrument driving mechanism as the mating interface. This may beneficially allow the endoscope to be quickly plugged into the instrument driving mechanism or robotic support without utilizing extra cables. Such type of electrical interface may also serve as a mechanical interface such that when the handle portion is plugged into the instrument driving mechanism, both mechanical and electrical coupling is established. Alternatively or in addition to, the instrument driving mechanism may provide a mechanical interface only. The handle portion may be in electrical communication with a modular wireless communication device or any other user device (e.g., portable/hand-held device or controller) for transmitting sensor data or receiving control signals.

In some cases, the handle portion 1409 may comprise one or more mechanical control modules such as lure 1411 for interfacing the irrigation system/aspiration system. In some cases, the handle portion may include lever/knob for articulation control. Alternatively, the articulation control may be located at a separate controller attached to the handle portion via the instrument driving mechanism.

The endoscope may be attached to a robotic support system or a hand-held controller via the instrument driving mechanism. The instrument driving mechanism may be provided by any suitable controller device (e.g., hand-held controller) that may or may not include a robotic system. The instrument driving mechanism may provide mechanical and electrical interface to the steerable catheter assembly 1400. The mechanical interface may allow the steerable catheter assembly 1400 to be releasably coupled to the instrument driving mechanism. For instance, the handle portion of the steerable catheter assembly can be attached to the instrument driving mechanism via quick install/release means, such as magnets, spring-loaded levels and the like. In some cases, the steerable catheter assembly may be coupled to or released from the instrument driving mechanism manually without using a tool.

In the illustrated example, the distal tip of the catheter or endoscope shaft is configured to be articulated/bent in two or more degrees of freedom to provide a desired camera view or control the direction of the endoscope. As illustrated in the example, imaging device (e.g., camera), position sensors (e.g., electromagnetic sensor) 1407 is located at the tip of the catheter or endoscope shaft 1405. For example, line of sight of the camera may be controlled by controlling the articulation of the active bending section 1403. In some instances, the angle of the camera may be adjustable such that the line of sight can be adjusted without or in addition to articulating the distal tip of the catheter or endoscope shaft. For example, the camera may be oriented at an angle (e.g., tilt) with respect to the axial direction of the tip of the endoscope with aid of an optical component.

The distal tip 1405 may be a rigid component that allow for positioning sensors such as electromagnetic (EM) sensors, imaging devices (e.g., camera) and other electronic components (e.g., LED light source) being embedded at the distal tip.

In real-time EM tracking, the EM sensor comprising of one or more sensor coils embedded in one or more locations and orientations in the medical instrument (e.g., tip of the endoscopic tool) measures the variation in the EM field created by one or more EM field generators positioned at a location close to a patient. The location information detected by the EM sensors is stored as EM data. The EM field generator (or transmitter), may be placed close to the patient to create a low intensity alternating magnetic field that the embedded sensor may detect. The alternating magnetic field induces small currents in the sensor coils of the EM sensor, which may be analyzed to determine the distance and angle between the EM sensor and the EM field generator. For example, the EM field generator may be positioned close to the patient torso during procedure to locate the EM sensor position in 3D space or may locate the EM sensor position and orientation in 5DOF or 6DOF. This may provide a visual guide to an operator when driving the bronchoscope towards the target site.

The endoscope may have a unique design in the elongate member. In some cases, the active bending section 1403, and the proximal shaft of the endoscope may consist of a single tube that incorporates a series of cuts (e.g., reliefs, slits, etc.) along its length to allow for improved flexibility, a desirable stiffness as well as the anti-prolapse feature (e.g., features to define a minimum bend radius).

As described above, the active bending section 1403 may be designed to allow for bending in two or more degrees of freedom (e.g., articulation). A greater bending degree such as 180-degrees and 270-degrees (or other articulation parameters for clinical indications) can be achieved by the unique structure of the active bending section. In some cases, a variable minimum bend radius along the axial axis of the elongate member may be provided such that an active bending section may comprise two or more different minimum bend radii.

The articulation of the endoscope may be controlled by applying force to the distal end of the endoscope via one or multiple pull wires. The one or more pull wires may be attached to the distal end of the endoscope. In the case of multiple pull wires, pulling one wire at a time may change the orientation of the distal tip to pitch up, down, left, right or any direction needed. In some cases, the pull wires may be anchored at the distal tip of the endoscope, running through the bending section, and entering the handle where they are coupled to a driving component (e.g., pulley). This handle pulley may interact with an output shaft from the robotic system.

In some embodiments, the proximal end or portion of one or more pull wires may be operatively coupled to various mechanisms (e.g., gears, pulleys, capstans, etc.) in the handle portion of the catheter assembly. The pull wire may be a metallic wire, cable or thread, or it may be a polymeric wire, cable or thread. The pull wire can also be made of natural or organic materials or fibers. The pull wire can be any type of suitable wire, cable, or thread capable of supporting various kinds of loads without significant deformation or breakage. The distal end/portion of one or more pull wires may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

The pull wires may be made of any suitable material such as stainless steel (e.g., SS316), metals, alloys, polymers, nylons, or biocompatible material. Pull wires may be a wire, cable, or a thread. In some embodiments, different pull wires may be made of different materials for varying the load bearing capabilities of the pull wires. In some embodiments, different sections of the pull wires may be made of different material to vary the stiffness or load bearing along the pull. In some embodiments, pull wires may be utilized for the transfer of electrical signals.

The proximal design may improve the reliability of the device without introducing extra cost allowing for a low-cost single-use endoscope. In another aspect of the invention, a single-use robotic endoscope is provided. The robotic endoscope may be a bronchoscope and can be the same as the steerable catheter assembly as described elsewhere herein. Traditional endoscopes can be complex in design and are usually designed to be re-used after procedures, which require thorough cleaning, dis-infection, or sterilization after each procedure. The existing endoscopes are often designed with complex structures to ensure the endoscopes can endure the cleaning, dis-infection, and sterilization processes. The provided robotic bronchoscope can be a single-use endoscope that may beneficially reduce cross-contamination between patients and infections. In some cases, the robotic bronchoscope may be delivered to the medical practitioner in a pre-sterilized package and are intended to be disposed of after a single-use.

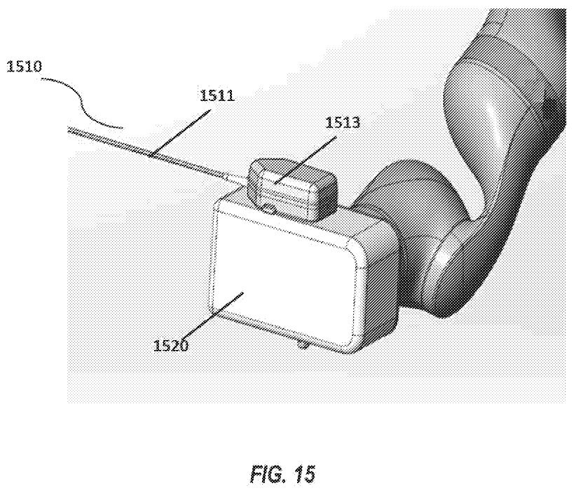

As shown in FIG. 15, a robotic bronchoscope 1510 may comprise a handle portion 1513 and a flexible elongate member 1511. In some embodiments, the flexible elongate member 1511 may comprise a shaft, steerable tip, and a steerable/active bending section. The robotic bronchoscope 1510 can be the same as the steerable catheter assembly as described in FIG. 14. The robotic bronchoscope may be a single-use robotic endoscope. In some cases, only the catheter may be disposable. In some cases, at least a portion of the catheter may be disposable. In some cases, the entire robotic bronchoscope may be released from the instrument driving mechanism and can be disposed of. In some cases, the bronchoscope may contain varying levels of stiffness along its shaft, as to improve functional operation. In some cases, a minimum bend radius along the shaft may vary.

The robotic bronchoscope can be releasably coupled to an instrument driving mechanism 1520. The instrument driving mechanism 1520 may be mounted to the arm of the robotic support system or to any actuated support system as described elsewhere herein. The instrument driving mechanism may provide mechanical and electrical interface to the robotic bronchoscope 1510. The mechanical interface may allow the robotic bronchoscope 1510 to be releasably coupled to the instrument driving mechanism. For instance, the handle portion of the robotic bronchoscope can be attached to the instrument driving mechanism via quick install/release means, such as magnets and spring-loaded levels. In some cases, the robotic bronchoscope may be coupled or released from the instrument driving mechanism manually without using a tool.

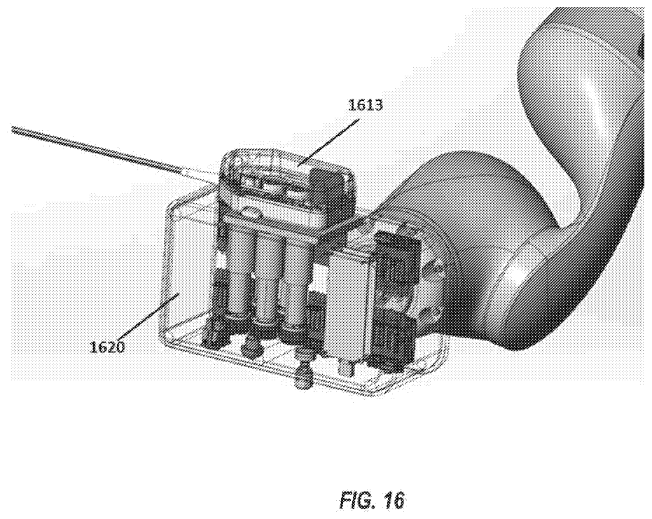

FIG. 16 shows an example of an instrument driving mechanism 1600B providing mechanical interface to the handle portion 1613 of the robotic bronchoscope. As shown in the example, the instrument driving mechanism 1600B may comprise a set of motors that are actuated to rotationally drive a set of pull wires of the flexible endoscope or catheter. The handle portion 1613 of the catheter assembly may be mounted onto the instrument drive mechanism so that its pulley assemblies or capstans are driven by the set of motors. The number of pulleys may vary based on the pull wire configurations. In some cases, one, two, three, four, or more pull wires may be utilized for articulating the flexible endoscope or catheter.

The handle portion may be designed to allow the robotic bronchoscope to be disposable at reduced cost. For instance, classic manual and robotic bronchoscopes may have a cable in the proximal end of the bronchoscope handle. The cable often includes illumination fibers, camera video cable, and other sensors fibers or cables such as electromagnetic (EM) sensors, or shape sensing fibers. Such complex cable can be expensive, adding to the cost of the bronchoscope. The provided robotic bronchoscope may have an optimized design such that simplified structures and components can be employed while preserving the mechanical and electrical functionalities. In some cases, the handle portion of the robotic bronchoscope may employ a cable-free design while providing a mechanical/electrical interface to the catheter.

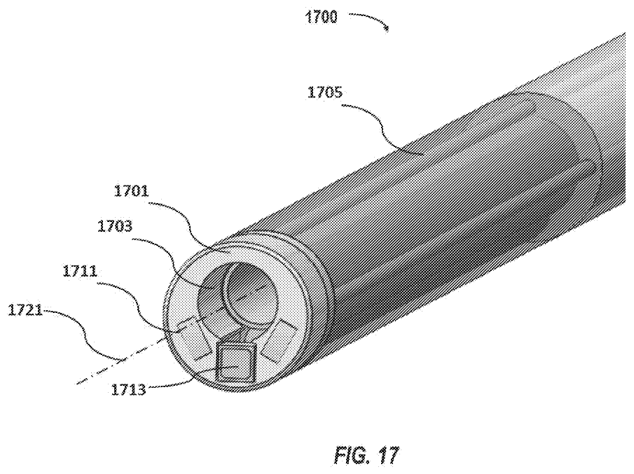

FIG. 17 shows an example of a distal tip 1700 of an endoscope. In some cases, the distal portion or tip of the catheter 1700 may be substantially flexible such that it can be steered into one or more directions (e.g., pitch, yaw). The catheter may comprise a tip portion, bending section, and insertion shaft. In some embodiments, the catheter may have variable bending stiffness along the longitudinal axis direction. For instance, the catheter may comprise multiple sections having different bending stiffness (e.g., flexible, semi-rigid, and rigid). The bending stiffness may be varied by selecting materials with different stiffness/rigidity, varying structures in different segments (e.g., cuts, patterns), adding additional supporting components or any combination of the above. In some embodiments, the catheter may have variable minimum bend radius along the longitudinal axis direction. The selection of different minimum bend radius at different location long the catheter may beneficially provide anti-prolapse capability while still allow the catheter to reach hard-to-reach regions. In some cases, a proximal end of the catheter needs not be bent to a high degree thus the proximal portion of the catheter may be reinforced with additional mechanical structure (e.g., additional layers of materials) to achieve a greater bending stiffness. Such design may provide support and stability to the catheter. In some cases, the variable bending stiffness may be achieved by using different materials during extrusion of the catheter. This may advantageously allow for different stiffness levels along the shaft of the catheter in an extrusion manufacturing process without additional fastening or assembling of different materials.

The distal portion of the catheter may be steered by one or more pull wires 1705. The distal portion of the catheter may be made of any suitable material such as co-polymers, polymers, metals, or alloys such that it can be bent by the pull wires. In some embodiments, the proximal end or terminal end of one or more pull wires 1705 may be coupled to a driving mechanism (e.g., gears, pulleys, capstan etc.) via the anchoring mechanism as described above.

The pull wire 1705 may be a metallic wire, cable, or thread, or it may be a polymeric wire, cable, or thread. The pull wire 1705 can also be made of natural or organic materials or fibers. The pull wire 1705 can be any type of suitable wire, cable, or thread capable of supporting various kinds of loads without significant deformation or breakage. The distal end or portion of one or more pull wires 1705 may be anchored or integrated to the distal portion of the catheter, such that operation of the pull wires by the control unit may apply force or tension to the distal portion which may steer or articulate (e.g., up, down, pitch, yaw, or any direction in-between) at least the distal portion (e.g., flexible section) of the catheter.

The catheter may have a dimension so that one or more electronic components can be integrated to the catheter. For example, the outer diameter of the distal tip may be around 4 to 4.4 millimeters (mm), and the diameter of the working channel may be around 2 mm such that one or more electronic components can be embedded into the wall of the catheter. However, it should be noted that based on different applications, the outer diameter can be in any range smaller than 4 mm or greater than 4.4 mm, and the diameter of the working channel can be in any range according to the tool dimensional or specific application.

The one or more electronic components may comprise an imaging device, illumination device, or sensors. In some embodiments, the imaging device may be a video camera 1713. The imaging device may comprise optical elements and image sensor for capturing image data. The image sensors may be configured to generate image data in response to a broad range of wavelengths of light or to specific wavelengths of light. A variety of image sensors may be employed for capturing image data such as complementary metal oxide semiconductor (CMOS) or charge-coupled device (CCD). The imaging device may be a low-cost camera. In some cases, the image sensor may be provided on a circuit board. The circuit board may be an imaging printed circuit board (PCB). The PCB may comprise a plurality of electronic elements for processing the image signal. For instance, the circuit for a CCD sensor may comprise A/D converters and amplifiers to amplify and convert the analog signal provided by the CCD sensor. Optionally, the image sensor may be integrated with amplifiers and converters to convert analog signal to digital signal such that a circuit board may not be required. In some cases, the output of the image sensor or the circuit board may be image data (digital signals) can be further processed by a camera circuit or processors of the camera. In some cases, the image sensor may comprise an array of optical sensors.

The illumination device may comprise one or more light sources 1711 positioned at the distal tip. The light source may be a light-emitting diode (LED), an organic LED (OLED), a quantum dot (QD), an array or combination of multiple LEDs, OLEDs, or QDs, or any other suitable light source. In some cases, the light source may include miniaturized LEDs for a compact design or Dual Tone Flash LED Lighting.

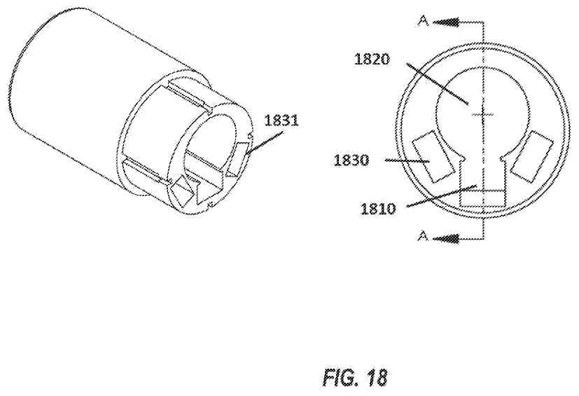

The imaging device and the illumination device may be integrated to the catheter. For example, the distal portion of the catheter may comprise suitable structures matching at least a dimension of the imaging device and the illumination device. The imaging device and the illumination device may be embedded into the catheter. FIG. 18 shows an example distal portion of the catheter with integrated imaging device and the illumination device. A camera may be located at the distal portion. The distal tip may have a structure to receive the camera, illumination device or the location sensor. For example, the camera may be embedded into a cavity 1810 at the distal tip of the catheter. The cavity 1810 may be integrally formed with the distal portion of the cavity and may have a dimension matching a length/width of the camera such that the camera may not move relative to the catheter. The camera may be adjacent to the working channel 1820 of the catheter to provide near field view of the tissue or the organs. In some cases, the attitude or orientation of the imaging device may be controlled by controlling a rotational movement (e.g., roll) of the catheter.

The power to the camera may be provided by a wired cable. In some cases, the cable wire may be in a wire bundle providing power to the camera as well as illumination elements or other circuitry at the distal tip of the catheter. The camera or light source may be supplied with power from a power source located at the handle portion via wires, copper wires, or via any other suitable means running through the length of the catheter. In some cases, real-time images or video of the tissue or organ may be transmitted to an external user interface or display wirelessly. The wireless communication may be WiFi, Bluetooth, RF communication or other forms of communication. In some cases, images or videos captured by the camera may be broadcasted to a plurality of devices or systems. In some cases, image or video data from the camera may be transmitted down the length of the catheter to the processors situated in the handle portion via wires, copper wires, or via any other suitable means. The image or video data may be transmitted via the wireless communication component in the handle portion to an external device/system. In some cases, the system may be designed such that no wires are visible or exposed to operators.

In conventional endoscopy, illumination light may be provided by fiber cables that transfer the light of a light source located at the proximal end of the endoscope, to the distal end of the robotic endoscope. In some embodiments of the disclosure, miniaturized LED lights may be employed and embedded into the distal portion of the catheter to reduce the design complexity. In some cases, the distal portion may comprise a structure 1430 having a dimension matching a dimension of the miniaturized LED light source. As shown in the illustrated example, two cavities 1430 may be integrally formed with the catheter to receive two LED light sources. For instance, the outer diameter of the distal tip may be around 4 to 4.4 millimeters (mm) and diameter of the working channel of the catheter may be around 2 mm such that two LED light sources may be embedded at the distal end. The outer diameter can be in any range smaller than 4 mm or greater than 4.4 mm, and the diameter of the working channel can be in any range according to the tool's dimensional or specific application. Any number of light sources may be included. The internal structure of the distal portion may be designed to fit any number of light sources.

In some cases, each of the LEDs may be connected to power wires which may run to the proximal handle. In some embodiment, the LEDs may be soldered to separated power wires that later bundle together to form a single strand. In some embodiments, the LEDs may be soldered to pull wires that supply power. In other embodiments, the LEDs may be crimped or connected directly to a single pair of power wires. In some cases, a protection layer such as a thin layer of biocompatible glue may be applied to the front surface of the LEDs to provide protection while allowing light emitted out. In some cases, an additional cover 1831 may be placed at the forwarding end face of the distal tip providing precise positioning of the LEDs as well as sufficient room for the glue. The cover 1831 may be composed of transparent material with similar refractive index to that of the glue so that the illumination light may not be obstructed.

### Examples of User Interfaces

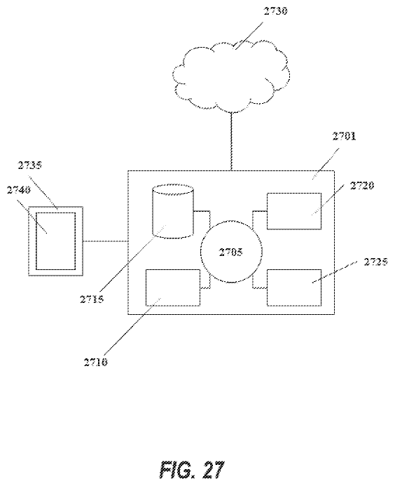

The systems, methods, and techniques described herein may be implemented at least in part with the use of a user interface that may be presented on a graphical user interface (e.g., the UI 2740 of FIG. 27). FIGS. 19-26 illustrate example user interfaces. At a high level, the user interfaces may be used for performing and interpreting tomosynthesis and augmented fluoroscopy.

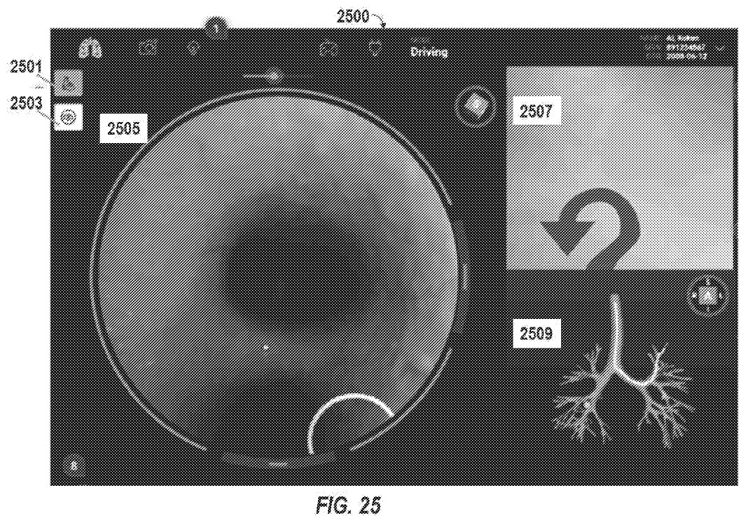

The graphical user interface (GUI) may allow a user to switch between multiple modes in a guided workflow. In some cases, a user interface for tomosynthesis may be accessible from user interfaces for driving or navigation. For example, when a user drives the endoscope via the driving or navigation interface 2500 as shown in FIG. 25, the user may choose to enter tomosynthesis mode by clicking on the icon 2501. For example, upon clicking on the icon 2501 switch to the tomosynthesis mode, a GUI (such as 1900 FIG. 19) of the tomosynthesis mode may be displayed. The tomosynthesis mode GUI may allow a user to return to the driving mode at any point such as by clicking the icon in the header 1901.

From the driving screen 2500, the user may continue viewing the camera feed 2505 from the bronchoscope and using the controller to drive through the lung. A user may choose to configure the driving GUI 2500 may adding or removing additional views. For example, the driving screen may be configured to display a virtual endoluminal view 2507, and virtual lungs 2509 which is a computer-generated 3D model of the lungs. The user may be permitted to add, remove, swap out one or more of other views such as the axial, coronal, and sagittal CTs and the like.

The virtual endoluminal view 2507 provides the user with a computer-recreated view of the camera feed along with a graphical element (e.g., ribbon) indicating the path to the currently selected target. In some cases, the path is also represented on the virtual lungs 2509. A user may switch to the tomosynthesis mode at any given time. For example, once the endoscope tip is within a biopsy-range of the target, the user may enable the tomosynthesis mode to help verify the relative distance to the lesion by clicking on the icon 2501. Details about the tomosynthesis operations and GUI are described later herein. After the tomosynthesis process is complete, the user may return to the driving screen 2500.

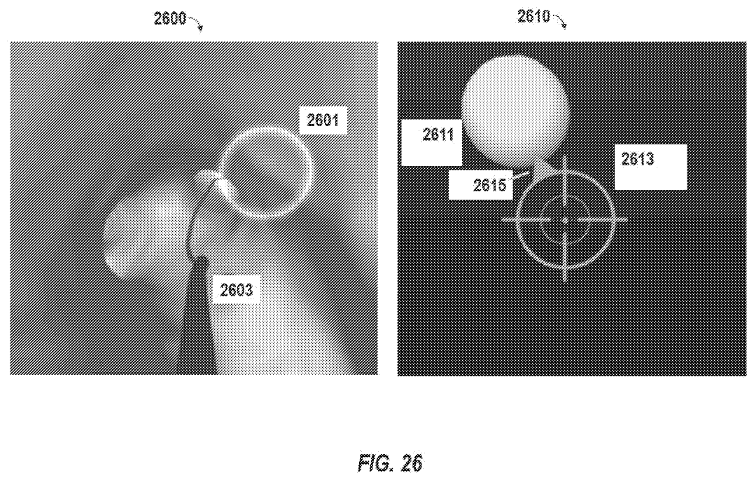

In some cases, upon completion of the tomosynthesis, the virtual endoluminal view may display a floating target based on the results of the tomography scan. FIG. 26 shows an example of the virtual endoluminal view 2600 displaying a target 2601 along with a graphical element 2603 (e.g., ribbon) indicating a path to the target. The angle of the target 2615 is displayed as seen from the point of view of the working channel, where a tool (e.g., needle instrument) will exit the bronchoscope. In some cases, an exit axis of the working channel may not be aligned to the axial axis of the endoscope distal tip (an example in FIG. 17 shows the exit axis 1721 of the working channel 1703). The point of view of the working channel may be based on a known dimension, structure or configuration of the distal tip (e.g., exit axis 1721 of the working channel with respect to the endoscope tip, the imaging device 1713) and/or a real-time orientation and location of the distal tip. The angle of the target 2615 relative to the exit axis of the working channel may be determined based at least in part on the layout of the working channel within the distal tip, a real-time location and orientation of the distal tip and location of the target. The target and the angle arrow 2615 may help to assist the user in lining up the tool with the lesion before taking a biopsy. The user may also choose to repeat the tomosynthesis process while the tool is expected to be in the lesion to increase confidence in the biopsy.

The virtual endoluminal panel displays a rendered view of the internal airways 2600. In some cases, the virtual endoluminal panel may allow a user to enter a targeting mode 2610. In some cases, once the user switches into targeting mode 2610, the rendered internal airways may disappear and the target 2611 may be displayed (e.g., depicted as a filled elliptical shape) in free space when the target is within a predetermined proximity range from the tip. The predetermined proximity range may be determined by the system or configurable by a user. In some cases, a graphical element (e.g., crosshair 2613, and arrow 2615) may appear in the center of the panel with a triangular shaped indicator around its edge to show the target's position relative to the direction the scope is facing.

In some cases, after tomosynthesis has been completed, the automated guided workflow may allow a user to adjust the position of the lesion (target) based on at least in part on a tomosynthesis calculation. In some cases, a tomosynthesis calculation may include a relationship between the position of the lesion and position of the scope tip. For instance, the position of the lesion may be automatically updated based on the relationship between the scope tip and lesion according to the tomosynthesis calculation. In some cases, a user may toggle the tomosynthesis calculated adjustments to the target via the graphical icon 2503 shown in the driving screen 2500 in FIG. 25. When the toggle is on, the position of the target in the virtual lung and virtual endoluminal panels may be adjusted or updated to reflect the calculations made by the tomosynthesis process based on the user-selected scope and lesion. When the toggle is off, such calculations may be disregarded and the position of the scope tip may solely rely on EM data and the position of the target may solely rely on the planned target on the CT scans. In alternative cases, a user may choose to adjust the position of the scope instead of or in addition to adjusting the location of the lesion/target.

In some cases, after tomosynthesis has been completed, an augmented fluoroscopy may be available in the fluoroscopic mode. A user may enable the augmented fluoroscopy such as via the toggle 2401 displayed within the user interface 2400 of a fluoroscopy panel to switch on the augmented fluoroscopy mode. The fluoroscopic view mode may be accessed from the driving mode during the entire navigation process. For example, a user may switch to the fluoroscopy view mode from the driving mode via the driving screen. The fluoroscopy view may provide real-time fluoroscopy images/video. The user interface 2400 of the fluoroscopy panel may display an augmented fluoroscopy feature allowing a user to enable/disable the augmentation to the fluoroscopy view. For instance, if the augmented fluoroscopy is toggled on (“Enabled”), an overlay of the target/lesion 2403 may be displayed on the fluoroscopy view. In some cases, the option to toggle on/off the augmented fluoroscopy may be available regardless the tomosynthesis is completed. If the augmented fluoroscopy is toggled on (“Enabled”) prior to completion of tomosynthesis (when a target location is not available), there may not be a display of the overlay of the target/lesion. The availability of the target/lesion information from the tomosynthesis can be obtained as described above. For example, lesion information may be broadcasted for the augmented fluoroscopy overlay through data contracts between the state machines as described above.

Existing endoscopic systems utilizing tomosynthesis techniques may not be compatible with any types of imaging apparatus (e.g., C-arm system). For example, current endoscopic systems may either be compatible with selected C-arm system or require cumbersome setting up for each C-arm system. The endoscopic systems herein employ an improved tomosynthesis algorithm as described above that can be compatible with any type of C-arm with minimum or reduced information about the C-arm system. For example, the system herein may provide a user interface allowing easy and convenient set up of C-arm systems.

FIG. 19 shows the example user interface 1900 of a tomosynthesis process dashboard. As illustrated, the user interface 1900 includes a header, a camera panel, a step indicator, instructions, visual guidance, an exit tomography function, and progression buttons. The user interface 1900 for tomosynthesis may be accessible from user interfaces for driving or navigation. Each of the header may remain present and the camera panel may remain visible for the entire tomosynthesis process. Users of the user interface 1900 may be guided through tomosynthesis by screens that may be broken down into a series of steps with an indication to the user of where in the tomosynthesis process they are currently in (see the step indicator). Within each screen, the instructions and the visual guidance in the form of images or videos may be displayed. At any point during the tomosynthesis process, the user may be able to exit the tomosynthesis screens of the user interface 1900 and return to the driving user interfaces. The progression buttons may also allow the user to navigate through the steps of the tomosynthesis process as necessary.

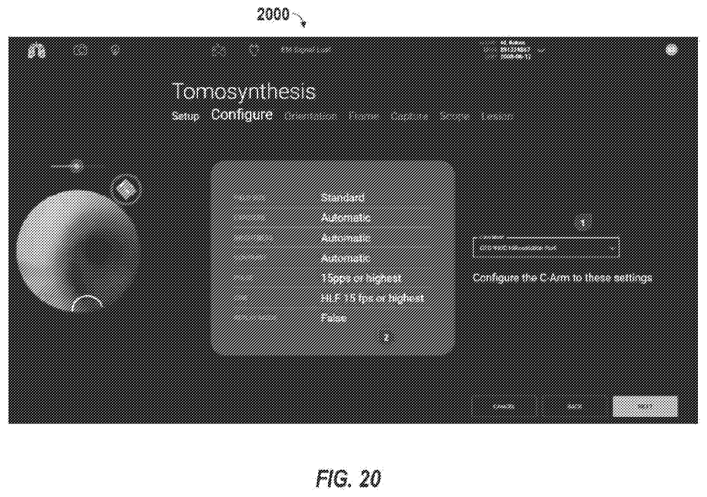

FIG. 20 shows an example user interface 2000 of a C-arm settings dashboard. As illustrated, the user interface 2000 includes a C-arm drop-down and C-arm settings. At the user interface 2000, the user may select the connected and compatible C-arm from the drop-down. Once the C-arm is selected, possible settings for that model of the C-arm may be displayed. The displayed settings may be default settings, previous settings, recommended setting, optimal settings, or the like. Once the C-arm settings are selected (e.g., by the user), the user may be instructed to adjust the C-arm to the selected settings.

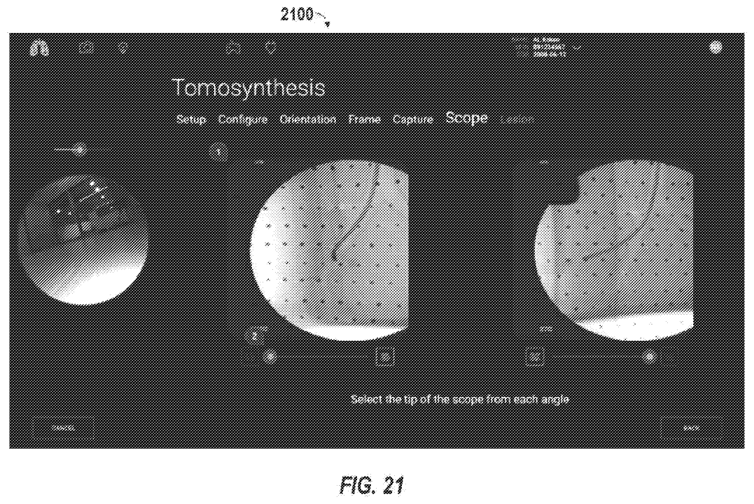

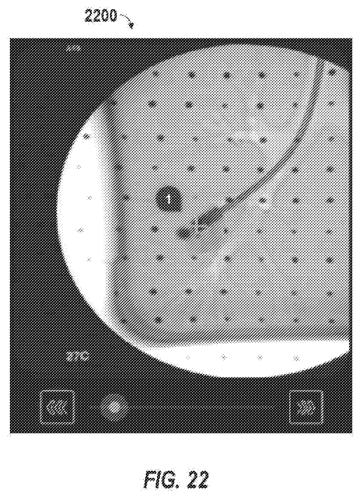

Upon setting up the imaging devices in the GUI (e.g., user interface 2000), the user may be guided to capture fluoroscopic images using the imaging device and may be guided to select a scope location via a GUI. FIG. 21 shows an example user interface 2100 of a scope selection dashboard. As illustrated, the user interface 2100 includes a fluoroscopy image and angle controls. At the user interface 2200, the fluoroscopy image may be displayed. The fluoroscopy image may be 2D images without augmentation. The user may be able to scroll through different angles of the scope captured from the C-arm using a slider shown in the user interface 2100 to choose one or more fluoroscopy images for selecting a location of the scope. For example, a user may click on the fluoroscopic image indicating a location of the scope tip. FIG. 22 shows an example user interface 2200 of a selection crosshair panel. The user interface 2200 may show a more detailed illustration of the fluoroscopy image of the user interface 2100. The user interface 2200 may include a selection crosshair. The user interface 2200 may display the selection cross hair upon the scope selection on the fluoroscopic image displayed within the user interface 2100 indicative of the location of the scope.

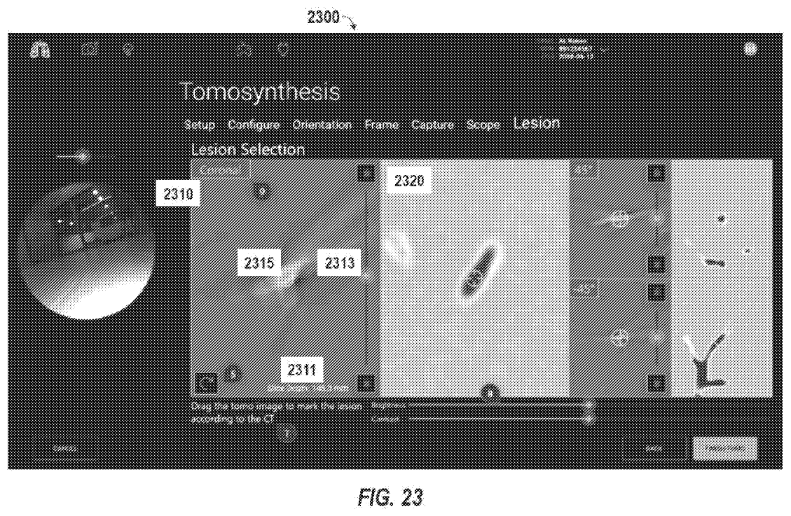

In some cases, upon selecting the location of the scope, the user may be guided to select a location for the target (e.g., lesion). FIG. 23 shows an example user interface 2300 of a lesion selection (target selection) dashboard. As illustrated, the user interface 2300 may display a reconstructed tomography 2310, CT Panels 2320, a selection crosshair 2315, a scrollbar 2313, a reset button, a depth indicator 2311, instructions, brightness and contrast controls, a view angle indicator. At the user interface 2300, once tomosynthesis scans have been captured, the user may be presented with pairs of CT 2320 and reconstructed tomography images 2310 from multiple orientations. As illustrated on the user interface 2300, within each set of images, the tomosynthesis images 2310 and the CT scans 2320 may be displayed with their corresponding view angle (e.g., view angle is indicated in the upper left corner). Crosshairs 2315 may be displayed in the user interface 2300 across all scans for a user to mark the lesion selection. The layers of each scan may be parsed via the scrollbar 2313 overlaid on the tomosynthesis image, with the depth of the view 2311 being displayed indicated within the image. The view may be able to be reset to its default by clicking on the reset button. The instructions as well as the brightness and contrast controls may be provided underneath the scans to guide the user through the process and allow them to adjust the image views as needed.

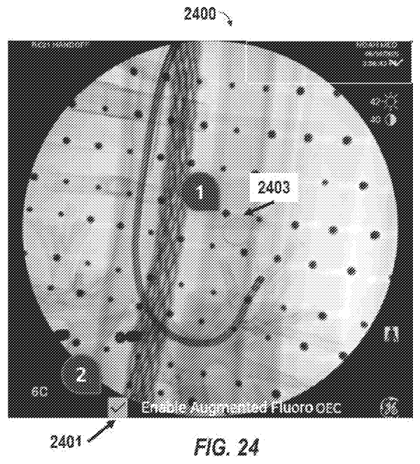

FIG. 24 shows an example user interface 2400 of an augmented fluoroscopy panel. As illustrated, the user interface 2400 includes a user selected lesion location indicator and an augmented fluoroscopy toggle. In some cases, after the tomosynthesis process has been completed, an overlay of the target location is available (e.g., based on the target location determined from the tomosynthesis and projected onto the 2D fluoroscopic image as described in FIG. 11 and elsewhere herein) which may enable an augmented fluoroscopic feature 2401. The user selected lesion location will be indicated on the fluoroscopy panel as an overlay on the user interface 2400. The overlay can be toggled (e.g., by the user) via the augmented fluoroscopy toggle. However, if the augmented fluoroscopy toggle is enabled and no overlay is available (e.g., the camera pose could not be reconstructed), then no change will be displayed on the fluoroscopy view.

### Computer System

The present disclosure provides computer systems that are programmed to implement methods of the disclosure. FIG. 27 shows a computer system 2701 that is programmed or otherwise configured to operate any method, system, process, or technique described herein (such as systems or methods of generating tomosynthesis reconstructions or augmented fluoroscopy, described herein). For example, the user interface 2740 may present one or more of the user interfaces described with respect to FIGS. 19-26.

The computer system 2701 can regulate various aspects of the present disclosure, such as, for example, techniques for tomosynthesis (e.g., tomosynthesis reconstruction) or fluoroscopy (e.g., augmented fluoroscopy). The computer system 2701 can be an electronic device of a user or a computer system that is remotely located with respect to the electronic device. The electronic device can be a mobile electronic device.

The computer system 2701 includes a central processing unit (CPU, also “processor” and “computer processor” herein) 2705, which can be a single core or multi core processor, or a plurality of processors for parallel processing. The computer system 2701 also includes memory or memory location 2710 (e.g., random-access memory, read-only memory, flash memory), electronic storage unit 2715 (e.g., hard disk), communication interface 2720 (e.g., network adapter) for communicating with one or more other systems, and peripheral devices 2725, such as cache, other memory, data storage or electronic display adapters. The memory 2710, storage unit 2715, interface 2720 and peripheral devices 2725 are in communication with the CPU 2705 through a communication bus (solid lines), such as a motherboard. The storage unit 2715 can be a data storage unit (or data repository) for storing data. The computer system 2701 can be operatively coupled to a computer network (“network”) 2730 with the aid of the communication interface 2720. The network 2730 can be the Internet, an internet or extranet, or an intranet or extranet that is in communication with the Internet. The network 2730 in some cases is a telecommunication or data network. The network 2730 can include one or more computer servers, which can enable distributed computing, such as cloud computing. The network 2730, in some cases with the aid of the computer system 2701, can implement a peer-to-peer network, which may enable devices coupled to the computer system 2701 to behave as a client or a server.

The CPU 2705 can execute instructions on computer-readable media, which can be embodied in a program or software. The instructions may be stored in a memory location, such as the memory 2710. The instructions can be directed to the CPU 2705, which can subsequently program or otherwise configure the CPU 2705 to implement methods of the present disclosure. Examples of operations performed by the CPU 2705 can include fetch, decode, execute, and writeback.

The CPU 2705 can be part of a circuit, such as an integrated circuit. One or more other components of the system 2701 can be included in the circuit. In some cases, the circuit is an application specific integrated circuit (ASIC).

The storage unit 2715 can store files, such as drivers, libraries, and saved programs. The storage unit 2715 can store user data, e.g., user preferences and user programs. The computer system 2701 in some cases can include one or more additional data storage units that are external to the computer system 2701, such as located on a remote server that is in communication with the computer system 2701 through an intranet or the Internet.

The computer system 2701 can communicate with one or more remote computer systems through the network 2730. For instance, the computer system 2701 can communicate with a remote computer system of a user (e.g., a medical device operator). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 2701 via the network 2730.

Methods as described herein can be implemented by way of machine (e.g., computer processor) executable code stored on an electronic storage location of the computer system 2701, such as, for example, on the memory 2710 or electronic storage unit 2715. The instructions may be code stored on the computer-readable media can be provided in the form of software. During use, the code can be executed by the processor 2705. In some cases, the code can be retrieved from the storage unit 2715 and stored on the memory 2710 for ready access by the processor 2705. In some situations, the electronic storage unit 2715 can be precluded, and machine-executable instructions are stored on memory 2710.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 2701, can be embodied in programming. Various aspects of the technology may be thought of as “products” or “articles of manufacture” typically in the form of computer-readable media storing instructions as code or associated data that is carried on or embodied in a type of computer-readable media. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable media” refer to any medium or media that participates in providing instructions to a processor for execution.

Hence, a computer-readable media, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code or data. Many of these forms of computer-readable media may be involved in carrying one or more sequences of one or more instructions to a processor for execution.

The computer system 2701 can include or be in communication with an electronic display 2735 that comprises a user interface (UI) 2740 for providing, for example, for tomosynthesis (e.g., tomosynthesis reconstruction) or fluoroscopy (e.g., augmented fluoroscopy) data, such as text, video, images, etc. Examples of UI's include, without limitation, a graphical user interface (GUI), a web-based user interface, or an Application Programming Interface (API). The UI 2740 may be, in some cases, also used for input via touchscreen capabilities.

Methods, systems, instructions, and techniques of the present disclosure can be implemented by way of one or more algorithms. An algorithm can be implemented by way of software upon execution by the central processing unit 2705. The algorithm may comprise: (a) providing a first graphical user interface (GUI) for a tomosynthesis mode and a second GUI for a fluoroscopic view mode for viewing a portion of the endoscopic device and a target within a subject; (b) receiving a sequence of fluoroscopic image frames containing the portion of the endoscopic device, a marker, and the target, where the sequence of fluoroscopic image frames correspond to various poses of an imaging system acquiring the sequence of fluoroscopic image frames; (c) upon switching to the tomosynthesis mode, i) performing a uniqueness check on the sequence of fluoroscopic image frames and ii) generating a reconstructed 3D tomosynthesis image based at least in part on the poses of the imaging system estimated using the marker; and (d) upon switching to the fluoroscopic view mode, i) generating an estimated pose of the imaging system associated with a fluoroscopic image frame from the sequence of fluoroscopic image frames based at least in part on the marker contained in the fluoroscopic image frame and ii) generating an overlay of the target displayed onto the fluoroscopic image frame based at least in part on the estimated pose. In some embodiments, the fluoroscopic images for the tomosynthesis and augmented fluoroscopy model may be acquired utilizing a Cone Beam CT (CBCT).

In some embodiments, the algorithm may implement operations including: (a) in a navigation mode of a graphical user interface (GUI), navigating the endoscopic device towards a target within a subject, the GUI displays a virtual view with visual elements to guide navigating the endoscopic device; (b) upon switching to a tomosynthesis mode of the GUI, i) receiving a sequence of fluoroscopic image frames containing a portion of the endoscopic device and the target, where the sequence of fluoroscopic image frames correspond to various poses of an imaging system acquiring the sequence of fluoroscopic image frames, ii) generating a reconstructed 3D tomosynthesis image based at least in part on the poses of the imaging system and iii) determining a location of the target based at least in part on the reconstructed 3D tomosynthesis image; and (c) upon switching to a fluoroscopic view mode of the GUI, i) obtaining a pose of the imaging system associated with a fluoroscopic image frame acquired in the fluoroscopic view mode, and ii) generating an overlay of the target displayed onto the fluoroscopic image frame based at least in part on the pose of the imaging system and the location of the target determined in (b).

In some embodiments, the virtual view in the navigation mode comprises upon determining a distal tip of the endoscopic device is within a predetermined proximity of the target, rendering a graphical representation of the target and an indicator indicative of an angle of the target relative to an exit axis of a working channel of the endoscopic device. In some embodiments, a location of the target displayed in the navigation mode is updated based on the location of the target determined in (b). In some embodiments, the poses of the imaging system in the tomosynthesis mode are estimated using a marker contained in the sequence of fluoroscopic image frames. In some embodiments, the poses of the imaging system in the tomosynthesis mode are measured by one or more sensors.

In some embodiments, the pose of the imaging system associated with the fluoroscopic image frame in the fluoroscopic view mode is estimated using a marker contained in the fluoroscopic image frame. In some cases, the marker has a 3D pattern. In some instances, the marker comprises a plurality of features placed on at least two different planes. In some cases, the marker has a plurality of features of different sizes arranged in a coded pattern. In some instances, the coded pattern comprises a plurality of sub-areas each has a unique pattern. In some instances, the pose of the imaging system is estimated by matching a patch of the plurality of features in the fluoroscopic image frame to the coded pattern.

In some embodiments, the pose of the imaging system associated with the fluoroscopic image frame in the fluoroscopic view mode is measured by one or more sensors. In some embodiments, in the tomosynthesis mode the sequence of fluoroscopic image frames are processed by performing a uniqueness check on the sequence of fluoroscopic image frames. In some cases, the uniqueness check comprises determining whether a fluoroscopic image frame from the sequence of fluoroscopic image frames is unique based at least in part on an intensity comparison.

### Example Method

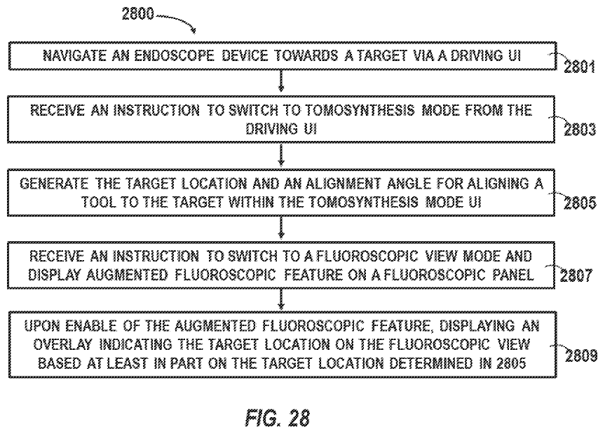

FIG. 28 illustrates an example method 2800 for presenting one or both of tomosynthesis reconstruction images or augmented fluoroscopy images in a guided workflow. The method may comprise: navigating an endoscope device towards a target via a driving UI 2801; receiving an instruction to switch to tomosynthesis mode from the driving UI 2803; generating the target location and an alignment angle for aligning a tool to the target within the tomosynthesis mode UI 2805; receiving an instruction to switch to a fluoroscopic view mode and displaying augmented fluoroscopic feature on a fluoroscopic panel 2807; and upon enable of the augmented fluoroscopic feature, displaying an overlay indicating the target location on the fluoroscopic view based at least in part on the target location determined in the tomosynthesis 2809.

For example, a user may navigate an endoscopic device towards a target via a first UI such as the driving UI as described above 2801; upon receiving an instruction to switch to a tomosynthesis imaging mode, providing a second UI displaying a tomosynthesis reconstruction 2803, where the tomosynthesis reconstruction is generated by: (i) acquiring one or more fluoroscopic images or 2D scans over a region of interest of a patient, and at least part of the fluoroscopic images over the region of interest includes first image data corresponding to a plurality of markers and the reconstructed tomosynthesis image comprises a plurality of tomosynthesis slices; displaying an indicator indicative of the target location and an angle indicator for aligning a tool to the target, where the target location and the angle is determined based at least in part on user input received via the second UI. The tomosynthesis image is reconstructed based on the fluoroscopic images and the plurality of markers.

The method may comprise receiving a user input to switch to a fluoroscopy mode. The fluoroscopy mode may provide a third UI displaying an augmented fluoroscopy feature allowing for enabling/disabling an augmented overlay to be displayed over the fluoroscopy view. The augmented fluoroscopic overlay is generated based at least in part on the target location identified in the tomosynthesis imaging. The third UI may be accessed from the first UI. In some embodiments, the fluoroscopic images for the tomosynthesis and fluoroscopy images for the fluoroscopic view may be acquired utilizing a Cone Beam CT (CBCT).

In some cases, upon completion of the tomosynthesis, the navigation mode UI or driving UI may be automatically updated. For example, a virtual endoluminal view of the driving UI may display a floating target based on the results of the tomography scan. The virtual endoluminal view can be the same as those illustrated in FIG. 26 where a target along with a graphical element (e.g., ribbon) indicating a path to the target is displayed. The angle of the target is also displayed as seen from the point of view of the working channel, where a tool (e.g., needle instrument) will exit the bronchoscope. The angle of the target relative to the exit axis of the working channel may be determined based at least in part on the layout of the working channel within the distal tip, a real-time location and orientation of the distal tip and location of the target obtained from the tomosynthesis result. The target and the angle arrow may help to assist the user in lining up the tool with the lesion before taking a biopsy. The user may also choose to repeat the tomosynthesis process while the tool is expected to be in the lesion to increase confidence in the biopsy.

The navigation mode UI or the driving UI may also provide a user a targeting mode as described in FIG. 26. A user may switch into targeting mode in which the rendered internal airways may disappear and the target may be displayed (e.g., depicted as a filled elliptical shape) in free space when the target is within a predetermined proximity range from the tip. The predetermined proximity range may be determined by the system or configurable by a user. In some cases, a graphical element (e.g., crosshair, and arrow) may appear in the center of the panel with a triangular shaped indicator around its edge to show the target's position relative to the direction the scope is facing. As escribed above, the visual indicator such as the location of the crosshair, the arrow may be determined based at least in part on the tomosynthesis result.

The method 2800 may implement one or more of the systems, methods, computer-readable media, techniques, processes, operations, or the like that are described herein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

It should be understood, that any reference herein to the term “or” is intended to mean an “inclusive or” or what is also known as a “logical OR”, wherein when used as a logic statement, the expression “A or B” is true if either A or B is true, or if both A and B are true, and when used as a list of elements, the expression “A, B or C” is intended to include all combinations of the elements recited in the expression, for example, any of the elements selected from the group consisting of A, B, C, (A, B), (A, C), (B, C), and (A, B, C); and so on if additional elements are listed. Furthermore, it should also be understood that the indefinite articles “a” or “an”, and the corresponding associated definite articles “the” or “said”, are each intended to mean one or more unless otherwise stated, implied, or physically impossible. Yet further, it should be understood that the expressions “at least one of A and B, etc.”, “at least one of A or B, etc.”, “selected from A and B, etc.” and “selected from A or B, etc.” are each intended to mean either any recited element individually or any combination of two or more elements, for example, any of the elements from the group consisting of “A”, “B”, and “A AND B together”, etc.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where values are described in the application and claims, unless otherwise stated the term “about” meaning within an acceptable error range for the particular value may be assumed.

It should be noted that various illustrative or suggested ranges set forth herein are specific to their example embodiments and are not intended to limit the scope or range of disclosed technologies, but, again, merely provide example ranges for frequency, amplitudes, etc. associated with their respective embodiments or use cases.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A computer-implemented method for an endoscopic device, comprising:
(a) in a navigation mode of a graphical user interface (GUI), navigating the endoscopic device towards a target within a subject, wherein the GUI displays a virtual view with visual elements to guide navigating the endoscopic device;
(b) upon switching to a tomosynthesis mode of the GUI, i) receiving a sequence of fluoroscopic image frames containing a portion of the endoscopic device and the target, wherein the sequence of fluoroscopic image frames correspond to various poses of an imaging system acquiring the sequence of fluoroscopic image frames, ii) generating a reconstructed 3D tomosynthesis image based at least in part on the poses of the imaging system and iii) determining a location of the target based at least in part on the reconstructed 3D tomosynthesis image; and
(c) upon switching to a fluoroscopic view mode of the GUI, i) obtaining a pose of the imaging system associated with a fluoroscopic image frame acquired in the fluoroscopic view mode, and ii) generating an overlay of the target displayed onto the fluoroscopic image frame based at least in part on the pose of the imaging system and the location of the target determined in (b).

2. The computer-implemented method of claim 1, wherein the virtual view in the navigation mode comprises upon determining a distal tip of the endoscopic device is within a predetermined proximity of the target, rendering a graphical representation of the target and an indicator indicative of an angle of the target relative to an exit axis of a working channel of the endoscopic device.

3. The computer-implemented method of claim 1, wherein a location of the target displayed in the navigation mode is updated based on the location of the target determined in (b).

4. The computer-implemented method of claim 1, wherein the poses of the imaging system in the tomosynthesis mode are estimated using a marker contained in the sequence of fluoroscopic image frames.

5. The computer-implemented method of claim 1, wherein the poses of the imaging system in the tomosynthesis mode are measured by one or more sensors.

6. The computer-implemented method of claim 1, wherein the pose of the imaging system associated with the fluoroscopic image frame in the fluoroscopic view mode is estimated using a marker contained in the fluoroscopic image frame.

7. The computer-implemented method of claim 6, wherein the marker has a 3D pattern.

8. The computer-implemented method of claim 7, wherein the marker comprises a plurality of features placed on at least two different planes.

9. The computer-implemented method of claim 7, wherein the marker has a plurality of features of different sizes arranged in a coded pattern.

10. The computer-implemented method of claim 9, wherein the coded pattern comprises a plurality of sub-areas each has a unique pattern.

11. The computer-implemented method of claim 9, wherein the pose of the imaging system is estimated by matching a patch of the plurality of features in the fluoroscopic image frame to the coded pattern.

12. The computer-implemented method of claim 1, wherein the pose of the imaging system associated with the fluoroscopic image frame in the fluoroscopic view mode is measured by one or more sensors.

13. The computer-implemented method of claim 1, wherein in the tomosynthesis mode, further comprising determining whether a fluoroscopic image frame from the sequence of fluoroscopic image frames is unique based at least in part on an intensity comparison.

14. A non-transitory computer-readable media storing instructions which, when executed by at least one processor, cause the at least one processor to perform operations comprising:
(a) in a navigation mode of a graphical user interface (GUI), navigating an endoscopic device towards a target within a subject, wherein the GUI displays a virtual view with visual elements to guide navigating the endoscopic device;
(b) upon switching to a tomosynthesis mode of the GUI, i) receiving a sequence of fluoroscopic image frames containing a portion of the endoscopic device and the target, wherein the sequence of fluoroscopic image frames correspond to various poses of an imaging system acquiring the sequence of fluoroscopic image frames, ii) generating a reconstructed 3D tomosynthesis image based at least in part on the poses of the imaging system and iii) determining a location of the target based at least in part on the reconstructed 3D tomosynthesis image; and
(c) upon switching to a fluoroscopic view mode of the GUI, i) obtaining a pose of the imaging system associated with a fluoroscopic image frame acquired in the fluoroscopic view mode, and ii) generating an overlay of the target displayed onto the fluoroscopic image frame based at least in part on the pose of the imaging system and the location of the target determined in (b).

15. The non-transitory computer-readable media of claim 14, wherein the virtual view in the navigation mode comprises upon determining a distal tip of the endoscopic device is within a predetermined proximity of the target, rendering a graphical representation of the target and an indicator indicative of an angle of the target relative to an exit axis of a working channel of the endoscopic device.

16. The non-transitory computer-readable media of claim 14, wherein a location of the target displayed in the navigation mode is updated based on the location of the target determined in (b).

17. The non-transitory computer-readable media of claim 14, wherein the poses of the imaging system in the tomosynthesis mode are estimated using a marker contained in the sequence of fluoroscopic image frames, and wherein the marker has a 3D pattern.

18. The non-transitory computer-readable media of claim 14, wherein the poses of the imaging system in the tomosynthesis mode are measured by one or more sensors.

19. The non-transitory computer-readable media of claim 14, wherein the pose of the imaging system associated with the fluoroscopic image frame in the fluoroscopic view mode is estimated using a marker contained in the fluoroscopic image frame and wherein the marker comprises a plurality of features placed on at least two different planes or wherein the marker has a plurality of features of different sizes arranged in a coded pattern.

20. The non-transitory computer-readable media of claim 14, wherein in the tomosynthesis mode, the one or more operations further comprise determining whether a fluoroscopic image frame from the sequence of fluoroscopic image frames is unique based at least in part on an intensity comparison.
